(19) United States
(12) Patent Application Publication
(10) Pub. No.: US 2024/0318146 A1
(43) Pub. Date: Sep. 26, 2024

(54) VASCULARIZED ORGANOIDS

(71) Applicant: CHILDREN’S HOSPITAL MEDICAL CENTER, Cincinnati, OH (US)

(72) Inventors: Mingxia GU, Cincinnati, OH (US); Yifei MIAO, Cincinnati, OH (US)

(21) Appl. No.: 18/684,587

(22) PCT Filed: Aug. 17, 2022
(86) PCT No.: PCT/US22/40633

§ 371 Date: Feb. 16, 2024

Related U.S. Application Data
(60) Provisional application No. 63/235,032, filed on Aug. 19, 2021.
(60) Provisional application No. 63/366,386, filed on Jun. 14, 2022.

(51) Int. Cl. C12N 5/071

(52) U.S. Cl. CPC C12N 5/0697; C12N 5/0679; C12N 5/0688; C12N 5/0691; C12N 2500/84; C12N 2501/15; C12N 2501/155; C12N 2501/16; C12N 2501/415; C12N 2506/45; C12N 2513/00

(57) ABSTRACT

Disclosed herein are compositions of organoids comprising a mesodermal vascular network and definitive endoderm derivatives. Also disclosed herein are methods making use of human pluripotent stem cells to differentiate cells of multiple germ layer lineages with proper organization. These organoids exhibit vasculature and can be used as models for studying endoderm-derived organogenesis and vascular interactions.

### CROSS-REFERENCE TO RELATED APPLICATIONS

This application claims the benefit of priority of U.S. Provisional Patent Application No. 63/235,032 filed Aug. 19, 2021, and U.S. Provisional Patent Application No. 63,366,386, filed Jun. 14, 2022.

### FIELD OF THE INVENTION

Aspects of the present disclosure relate generally to organoid compositions comprising a differentiated vascular network, methods of making and use thereof.

### BACKGROUND

The organ-specific characteristics of vascular endothelial cells (ECs) have long been recognized. Depending on the physical and biochemical property of the residing organ, the estimated 6×10¹¹ ECs in the human body develop remarkable heterogeneity in morphology, function, and molecular signatures. While ECs of arteries and veins form a continuous layer connected by tight junctions, capillary ECs could be continuous, fenestrated, or discontinuous, presumably to meet the distinct physiological needs of each tissue. For instance, the blood-brain barrier (BBB) consists of tightly linked ECs to restrict paracellular diffusion, whereas permeable fenestrations in liver sinusoidal ECs facilitate exchange of solutes. Recent single-cell analyses have reinforced the notion of EC heterogeneity in both human and mouse, and further transcriptionally and functionally identified distinct EC subpopulations even within the same vascular bed. In most vertebrates, cell specialization occurs in response to a combination of intrinsic (e.g., cellular ontogeny) and extrinsic (e.g., surrounding microenvironment) factors that drive the acquisition of the unique features. However, it has remained unclear what is driving EC fate determination during organogenesis.

### SUMMARY

Disclosed herein are methods of producing mesendoderm spheroids comprising both definitive endoderm and lateral plate mesoderm. In some embodiments, the methods comprise a) contacting pluripotent stem cells with a TGF-b pathway activator, a BMP pathway activator, and a Wnt pathway activator for about 24 hours to about 48 hours; and b) contacting the cells of step a) with a TGF-b pathway activator and a BMP pathway activator, without a Wnt pathway activator, for about 24 hours to about 72 hours, thereby differentiating the pluripotent stem cells to mesendoderm spheroids comprising both FOXFA2+ definitive endoderm and HAND1+ lateral plate mesoderm. In some embodiments, the cells of step a) and/or b) are further contacted with a serum supplement, optionally fetal bovine serum.

Also disclosed herein are methods of producing foregut spheroids from mesendoderm spheroids disclosed herein. In some embodiments, the methods comprise contacting mesendoderm spheroids with a BMP pathway inhibitor, a TGF-b pathway inhibitor, and optionally a Hedgehog pathway activator, for a period of time sufficient to differentiate the mesendoderm spheroids to foregut spheroids comprising SOX2+/FOXA2+ foregut epithelium and FOXF1+ splanchnic mesoderm.

Also disclosed herein are methods of producing mid/hindgut spheroids from mesendoderm spheroids disclosed herein. In some embodiments, the methods comprise contacting mesendoderm spheroids with a Wnt pathway activator and an FGF pathway activator for a period of time sufficient to differentiate the mesendoderm spheroids to mid/hindgut spheroids comprising CDX2+/FOXA2+ mid/hindgut epithelium and FOXF1+ splanchnic mesoderm.

Also disclosed herein are methods of producing ventral anterior foregut spheroids. In some embodiments, the methods comprise contacting any one of the foregut spheroids disclosed herein with retinoic acid for a period of time sufficient to differentiate the foregut spheroids to ventral anterior foregut spheroids.

Also disclosed herein are methods of producing vascularized lung organoids (vLuO). In some embodiments, the methods comprise contacting ventral anterior foregut spheroids with a Wnt pathway activator, a BMP pathway activator, and VEGF, and optionally a retinoic acid pathway activator, for a period of time sufficient to differentiate the ventral anterior foregut spheroids into vLuO, wherein the vLuO comprises TTF1/NKX2-1+ distal lung epithelial progenitors, is positive for SOX9, exhibits branching morphogenesis, and receives FGF10 signaling secreted by distal mesenchyme derived from FOXF1+ splanchnic mesoderm.

Also disclosed herein are methods of producing vascularized proximal lung organoids. In some embodiments, the methods comprise a) contacting ventral anterior foregut spheroids with a Wnt pathway activator, a BMP pathway activator, and VEGF, and optionally a retinoic acid pathway activator, for a period of time sufficient to differentiate the ventral anterior foregut spheroids into lung progenitors, and b) contacting the lung progenitors with a proximal lung specification medium comprising one or more FGF pathway activators and VEGF for a period of time sufficient to differentiate the lung progenitors to vascularized proximal lung organoids; wherein the one or more FGF pathway activators are provided at a concentration that is greater than that used to produce distal lung organoids.

Also disclosed herein are methods of producing vascularized small intestine organoids (vHIO). In some embodiments, the methods comprise contacting mid/hindgut spheroids with: 1) a BMP pathway inhibitor and VEGF, and optionally with R-spondin and EGF, for a first period of time; and 2) VEGF, and optionally EGF, for a second period of time, thereby differentiating the mid/hindgut spheroids into vHIO, wherein the vHIO expresses CDX2, GATA4, and CDH17, is negative for SOX2, and comprises a CD31+ vascular bed.

Also disclosed herein are methods of producing vascularized colonic organoids (vHCO). In some embodiments, the methods comprise contacting mid/hindgut spheroids with: 1) a BMP pathway activator and VEGF, and optionally EGF, for a first period of time; and 2) VEGF, and optionally EGF, for a second period of time, thereby differentiating the mid/hindgut spheroids into vHCO, wherein the vHCO expresses SATB2 and CDH17, and comprises a CD31+ vascular bed.

Also disclosed herein are mesendoderm spheroids, foregut spheroids, mid/hindgut spheroids, ventral anterior foregut spheroids, vLuO, vascularized proximal lung organoids, vHIO, and vHCO produced according to any of the methods disclosed herein. In some embodiments, the foregut spheroids, mid/hindgut spheroids, and ventral anterior foregut spheroids disclosed herein comprise endothelial progenitors.

Exemplary embodiments of the present disclosure are provided in the following numbered embodiments:

1. A method of producing mesendoderm spheroids comprising both definitive endoderm and lateral plate mesoderm, comprising:
a) contacting pluripotent stem cells with a TGF-b pathway activator, a BMP pathway activator, and a Wnt pathway activator for about 24 hours to about 48 hours; and
b) contacting the cells of step a) with a TGF-b pathway activator and a BMP pathway activator, without a Wnt pathway activator, for about 24 hours to about 72 hours; thereby differentiating the pluripotent stem cells to mesendoderm spheroids comprising both FOXA2+ definitive endoderm and HAND1+ lateral plate mesoderm, wherein the cells of step a) and/or b) are further contacted with a serum supplement, optionally fetal bovine serum.

2. The method of embodiment 1, wherein the lateral plate mesoderm surrounds the definitive endoderm in the mesendoderm spheroids.

3. The method of embodiment 1 or 2, wherein step a) is performed for about 26, 27, 28, 29, or 30 hours, optionally about 28 hours.

4. The method of any one of embodiments 1-3, wherein step b) is performed for about 42, 43, 44, 45, 46, 47, 48, 49, 50, 51, 52, 53, 54, 55, 56, 57, 58, 59, or 60 hours, optionally about 44 hours.

5. The method of any one of embodiments 1-4, wherein the TGF-b pathway activator of step a) and/or b) is TGF-beta 1, TGF-beta 2, TGF-beta 3, Activin A, Activin B, Nodal, a BMP, IDE1, IDE2, or any combination thereof, optionally Activin A.

6. The method of any one of embodiments 1-5, wherein the TGF-b pathway activator of step a) and/or b) is provided at a concentration of about 50, 60, 70, 80, 90, 100, 110, 120, 130, 140, or 150 ng/mL, or any concentration within a range defined by any two of the aforementioned concentrations.

7. The method of any one of embodiments 1-6, wherein the BMP pathway activator of step a) and/or b) is BMP1, BMP2, BMP3, BMP4, BMP5, BMP6, BMP7, BMP8a, BMP8b, BMP10, BMP11, BMP15, IDE1, IDE2, or any combination thereof, optionally BMP4.

8. The method of any one of embodiments 1-7, wherein the BMP pathway activator of step a) and/or b) is provided at a concentration of about 10, 20, 30, 40, 50, 60, 70, 80, 90, or 100 ng/mL, or any concentration within a range defined by any two of the aforementioned concentrations.

9. The method of any one of embodiments 1-8, wherein the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB216763, SB 415286, aloisine, indirubin, alsterpaullone, kenpaullone, lithium chloride, TDZD 8, TWS119, or any combination thereof, optionally CHIR99021.

10. The method of any one of embodiments 1-9, wherein the Wnt pathway activator is provided at a concentration of about 1, 2, 3, 4, 5, 6, 7, 8, 9, 10, 11, 12, 13, 14, 15, 16, 17, 18, 19, 20, 21, 22, 23, or 24 μM, or any concentration within a range defined by any two of the aforementioned concentrations, optionally 9, 10, 11, or 12 μM.

11. The method of any one of embodiments 1-10, wherein prior to the contacting of step a), the pluripotent stem cells are in the form of spheroids.

12. The method of claim 11, wherein the pluripotent stem cells are formed into spheroids by aggregation, optionally in an aggregation well, optionally wherein the number of cells aggregated is, or is about 200-4000, 300-3000, 500-2000, 600-1500, or 750-1250 cells.

13. The method of any one of embodiments 1-12, wherein for the duration of steps a) and b), the pluripotent stem cells are kept in suspension, optionally with shaking, and/or not as a monolayer.

14. The method of any one of embodiments 1-13, wherein the FOXA2+ definitive endoderm and the HAND1+ lateral plate mesoderm are approximately at a 1:1 ratio.

15. Mesendoderm spheroids prepared according to the methods of any one of embodiments 1-14.

16. A method of producing foregut spheroids from mesendoderm spheroids, comprising contacting mesendoderm spheroids with a BMP pathway inhibitor, a TGF-b pathway inhibitor, and optionally a Hedgehog pathway activator, for a period of time sufficient to differentiate the mesendoderm spheroids to foregut spheroids comprising SOX2+/FOXA2+ foregut epithelium and FOXF1+ splanchnic mesoderm.

17. The method of embodiment 16, wherein the period of time sufficient to differentiate the mesendoderm spheroids to foregut spheroids is about 2 days to 6 days, such as about 2, 3, 4, 5, or 6 days.

18. The method of embodiment 16 or 17, wherein the BMP pathway inhibitor is Noggin, Dorsomorphin, RepSox, LY364947, LDN193189, follistatin, chordin, or any combination thereof, optionally Noggin.

19. The method of any one of embodiments 16-18, wherein the BMP pathway inhibitor is provided at a concentration of about 50, 60, 70, 80, 90, 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations.

20. The method of any one of embodiments 16-19, wherein the TGF-b pathway inhibitor is A8301, RepSox, LY365947, SB431542, or any combination thereof, optionally SB431542.

21. The method of any one of embodiments 16-20, wherein the TGF-b pathway inhibitor is provided at a concentration of about 1, 2, 3, 4, 5, 6, 7, 8, 9, 10, 11, 12, 13, 14, 15, 16, 17, 18, 19, or 20 μM, or any concentration within a range defined by any two of the aforementioned concentrations, optionally 10 μM.

22. The method of any one of embodiments 16-21, wherein the Hedgehog pathway activator is Smoothened agonist (SAG).

23. The method of any one of embodiments 16-22, wherein the Hedgehog pathway activator is provided at a concentration of about 0.5, 1, 2, 3, 4, or 5 μM, or any concentration within a range defined by any two of the aforementioned concentrations, optionally 1 μM.

24. The method of any one of embodiments 16-23, further comprising contacting the mesendoderm spheroids with vascular endothelial growth factor (VEGF) to generate a CD31+ vascular network surrounding the SOX2+/FOXA2+ foregut epithelium.

25. The method of any one of embodiments 16-24, further comprising contacting the mesendoderm spheroids with a retinoic acid pathway activator, optionally wherein the retinoic acid pathway activator is retinoic acid, all-trans retinoic acid, 9-cis retinoic acid, CD437, EC23, BS 493, TTNPB, AM580, or any combination thereof.

26. The method of any one of embodiments 16-25, wherein the mesendoderm spheroids are not contacted with a Wnt pathway activator and/or an FGF pathway activator.

27. A method of producing mid/hindgut spheroids from mesendoderm spheroids, comprising contacting mesendoderm spheroids with a Wnt pathway activator and an FGF pathway activator for a period of time sufficient to differentiate the mesendoderm spheroids to mid/hindgut spheroids comprising CDX2+/FOXA2+ mid/hindgut epithelium and FOXF1+ splanchnic mesoderm.

28. The method of embodiment 27, wherein the period of time sufficient to differentiate the mesendoderm spheroids to mid/hindgut spheroids is about 2 days to 6 days.

29. The method of embodiment 27 or 28, wherein the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, TWS119, or any combination thereof, optionally CHIR99021.

30. The method of any one of embodiments 27-29, wherein the Wnt pathway activator is provided at a concentration of about 1, 2, 3, 4, 5, 6, 7, 8, 9, or 10 μM, or any concentration within a range defined by any two of the aforementioned concentrations.

31. The method of any one of embodiments 27-30, wherein the FGF pathway activator is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, FGF23, or any combination thereof, optionally FGF4.

32. The method of any one of embodiments 27-31, wherein the FGF pathway activator is provided at a concentration of about 100, 200, 300, 400, 500, 600, 700, 800, 900, or 1000 ng/mL, or any concentration within a range defined by any two of the aforementioned concentrations.

33. The method of any one of embodiments 27-32, further comprising contacting the mesendoderm spheroids with vascular endothelial growth factor (VEGF) to generate a CD31+ vascular network surrounding the CDX2+/FOXA2+ mid/hindgut epithelium.

34. The method of any one of embodiments 16-33, wherein the VEGF is provided at a concentration of about 10, 20, 30, 40, 50, 60, 70, 80, 90, or 100 ng/mL, or any concentration within a range defined by any two of the aforementioned concentrations.

35. The method of any one of embodiments 16-34, wherein the mesendoderm spheroids are the mesendoderm spheroids of embodiment 15.

36. Foregut spheroids produced according to the method of any one of embodiments 16-26, 34-35.

37. Mid/hindgut spheroids produced according to the method of any one of embodiments 27-35.

38. A method of producing ventral anterior foregut spheroids, comprising contacting the foregut spheroids producing according to the method of any one of embodiments 16-26, 34-35 with retinoic acid for a period of time sufficient to differentiate the foregut spheroids to ventral anterior foregut spheroids.

39. The method of embodiment 38, wherein the period of time sufficient to differentiate the foregut spheroids to ventral anterior foregut spheroids is about 1 day.

40. A method of producing vascularized distal lung organoids (vLuO), comprising:
a) contacting ventral anterior foregut spheroids with a Wnt pathway activator, a BMP pathway activator, and VEGF, and optionally a retinoic acid pathway activator, for a period of time sufficient to differentiate the ventral anterior foregut spheroids into lung progenitors, and
b) contacting the lung progenitors with a distal lung specification medium comprising a Wnt pathway activator, one or more FGF pathway activators, and VEGF, for a period of time sufficient to differentiate the lung progenitors to vLuO;
wherein the vLuO comprises TTF1/NKX2-1+ distal lung epithelial progenitors, is positive for SOX9, exhibits branching morphogenesis, and receives FGF10 signaling secreted by distal mesenchyme derived from FOXF1+ splanchnic mesoderm.

41. The method of embodiment 40, wherein the ventral anterior foregut spheroids comprise a CD31+ vascular network surrounding the SOX2+/FOXA2+ foregut epithelium.

42. The method of embodiment 40 or 41, wherein the ventral anterior foregut spheroids are the ventral anterior foregut spheroids produced by the method of embodiment 38 or 39.

43. The method of any one of embodiments 40-42, wherein the ventral anterior foregut spheroids of step a) are embedded in a basement membrane matrix and are contacted in a stationary culture for a first period of time and in a culture with shaking for a second period of time, wherein the shaking for the second period of time promotes circulation of nutrition and promotes vascularization.

44. The method of embodiment 43, wherein the first period of time is 1, 2, 3, 4, or 5 days, optionally 3 days, and/or the second period of time is 5, 6, 7, 8, 9, or 10 days, optionally 7 days.

45. The method of any one of embodiments 40-44, wherein the distal lung specification medium further comprises dexamethasone, cAMP, and 3-isobutyl-1-methylxanthine (IBMX).

46. The method of any one of embodiments 40-45, wherein the Wnt pathway activator of step a) and/or step b) is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, TWS119, or any combination thereof, optionally CHIR99021.

47. The method of any one of embodiments 40-46, wherein the Wnt pathway activator of step a) and/or step b) is provided at a concentration of about 1, 2, 3, 4, 5, 6, 7, 8, 9, or 10 μM, or any concentration within a range defined by any two of the aforementioned concentrations, optionally 3 μM.

48. The method of any one of embodiments 40-47, wherein the one or more FGF pathway activators are selected from FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, FGF23, or any combination thereof, optionally FGF7 and FGF10.

49. The method of any one of embodiments 40-48, wherein the one or more FGF pathway activators are each provided at a concentration of about 5, 6, 7, 8, 9, 10, 11, 12, 13, 14, or 15 ng/mL, or any concentration within a range defined by any two of the aforementioned concentrations, optionally 10 ng/mL.

50. The method of any one of embodiments 40-49, wherein the BMP pathway activator is BMP1, BMP2, BMP3, BMP4, BMP5, BMP6, BMP7, BMP8a, BMP8b, BMP10, BMP11, BMP15, IDE1, IDE2, or any combination thereof, optionally BMP4.

51. The method of any one of embodiments 40-50, wherein the BMP pathway activator is provided at a concentration of about 10, 20, 30, 40, 50, 60, 70, 80, 90, or 100 ng/mL, or any concentration within a range defined by any two of the aforementioned concentrations.

52. The method of any one of embodiments 40-51, wherein the retinoic acid pathway activator is retinoic acid, all-trans retinoic acid, 9-cis retinoic acid, CD437, EC23, BS 493, TTNPB, AM580, or any combination thereof, optionally all-trans retinoic acid (ATRA).

53. The method of any one of embodiments 40-52, wherein the VEGF is provided at a concentration of about 10, 20, 30, 40, 50, 60, 70, 80, 90, or 100 ng/mL, or any concentration within a range defined by any two of the aforementioned concentrations.

54. The vLuO produced according to the method of any one of claims 40-53, optionally wherein the vLuO is positive for one or more human lung EC markers selected from the group consisting of FENDRR, NCKAP5, HPGD, KIT, and PDE3B.

55. The vLuO of embodiment 54, comprising a FOXF1 mutation, optionally for use as a disease model for alveolar capillary dysplasia (ACD), misalignment of pulmonary vein (MPV), and/or pulmonary lymphangiectasia.

56. A method of producing vascularized proximal lung organoids, comprising:
a) contacting ventral anterior foregut spheroids with a Wnt pathway activator, a BMP pathway activator, and VEGF for a period of time sufficient to differentiate the ventral anterior foregut spheroids into lung progenitors, and
b) contacting the lung progenitors with a proximal lung specification medium comprising one or more FGF pathway activators and VEGF for a period of time sufficient to differentiate the lung progenitors to vascularized proximal lung organoids;
wherein the one or more FGF pathway activators are provided at a concentration that is greater than that used to produce distal lung organoids.

57. The method of embodiment 56, wherein the ventral anterior foregut spheroids comprise a CD31+ vascular network surrounding the SOX2+/FOXA2+ foregut epithelium.

58. The method of embodiment 56 or 57, wherein the ventral anterior foregut spheroids are the ventral anterior foregut spheroids produced by the method of embodiment 36 or 37.

59. The method of any one of embodiments 56-58, wherein the one or more FGF pathway activators are selected from FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, FGF23, or any combination thereof, optionally FGF2 and FGF10.

60. The method of any one of embodiments 56-59, wherein the one or more FGF pathway activators are each provided at a concentration of about 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations.

61. The method of any one of embodiments 56-60, wherein the proximal lung specification medium further comprises dexamethasone, cAMP, and IMBX.

62. The method of any one of embodiments 56-61, wherein the ventral anterior foregut spheroids are not contacted with a Wnt pathway activator.

63. The vascularized proximal lung organoids produced by the method of any one of embodiments 56-62.

64. The method of any one of embodiments 40-53 or 56-62, wherein the ventral anterior foregut cells are embedded in a lung-specific extracellular matrix during the contacting steps, optionally wherein the lung-specific extracellular matrix is isolated from human lung tissue.

65. The vLuO of embodiment 54 or 55, or the vascularized proximal lung organoids of embodiment 63, wherein the vLuO or the vascularized proximal lung organoids are embedded in a lung-specific extracellular matrix, optionally wherein the lung-specific extracellular matrix is isolated from human lung tissue.

66. The method of any one of embodiments 40-53 or 56-62, further comprising contacting the ventral anterior foregut spheroids with midkine (MDK), semaphorin-3C (SEMA3C), growth/differentiation factor-15 (GDF15), or any combination thereof.

67. A method comprising contacting the vLuO of embodiment 54 or 55, or the vascularized proximal lung organoids of embodiment 63 with a perfusion system.

68. A method of producing vascularized small intestine organoids (vHIO), comprising contacting mid/hindgut spheroids with:
1) a BMP pathway inhibitor and VEGF, and optionally with R-spondin and EGF, for a first period of time; and
2) VEGF, and optionally EGF, for a second period of time;
thereby differentiating the mid/hindgut spheroids into vHIO, wherein the vHIO expresses CDX2, GATA4, and CDH17, is negative for SOX2, and comprises a CD31+ vascular bed.

69. The method of embodiment 68, wherein the BMP pathway inhibitor is Noggin, Dorsomorphin, RepSox, LY364947, LDN193189, follistatin, chordin, or any combination thereof, optionally Noggin.

70. The method of embodiment 68 or 69, wherein the BMP pathway inhibitor is provided at a concentration of about 100, 110, 120, 130, 140, 150, 160, 170, 180, 190, or 200 ng/mL, or any concentration within a range defined by any two of the aforementioned concentrations.

71. The method of any one of embodiments 68-70, wherein the VEGF is provided at a concentration of about 10, 20, 30, 40, 50, 60, 70, 80, 90, or 100 ng/mL, or any concentration within a range defined by any two of the aforementioned concentrations.

72. The method of any one of embodiments 68-71, wherein the mid/hindgut spheroids are embedded in a basement membrane matrix, step 1) is carried out in a stationary culture, and step 2) is carried out in a culture with shaking.

73. The method of any one of embodiments 68-72, wherein the first period of time is 1, 2, 3, 4, or 5 days, optionally 3 days, and/or the second period of time is at least 10, 11, 12, 13, 14, 15, 16, 17, 18, 19, or 20 days.

74. The method of any one of embodiments 68-73, wherein the mid/hindgut spheroids comprise a CD31+ vascular network surrounding the CDX2+/FOXA2+ mid/hindgut epithelium.

75. The method of any one of embodiments 68-74, wherein the mid/hindgut spheroids are the mid/hindgut spheroids of embodiment 37.

76. A method of producing a vascularized colonic organoid (vHCO), comprising contacting mid/hindgut spheroids with;
1) a BMP pathway activator and VEGF, and optionally EGF, for a first period of time; and
2) VEGF, and optionally EGF, for a second period of time;
thereby differentiating the mid/hindgut spheroids into vHCO, wherein the vHCO expresses SATB2 and CDH17, and comprises a CD31+ vascular bed.

77. The method of embodiment 76, wherein the BMP pathway activator is BMP1, BMP2, BMP3, BMP4, BMP5, BMP6, BMP7, BMP8a, BMP8b, BMP10, BMP11, BMP15, IDE1, IDE2, or any combination thereof, optionally BMP2.

78. The method of embodiment 76 or 77, wherein the BMP pathway activator is provided at a concentration of about 50, 60, 70, 80, 90, 100, 110, 120, 130, 140, or 150 ng/mL, or any concentration within a range defined by any two of the aforementioned concentrations, optionally 100 ng/mL.

79. The method of any one of embodiments 68-78, wherein the VEGF is provided at a concentration of about 10, 20, 30, 40, 50, 60, 70, 80, 90, or 100 ng/mL, or any concentration within a range defined by any two of the aforementioned concentrations.

80. The method of any one of embodiments 68-79, wherein the mid/hindgut spheroids are embedded in a basement membrane matrix, step 1) is carried out in a stationary culture, and step 2) is carried out in a culture with shaking.

81. The method of any one of embodiments 68-80, wherein the first period of time is 1, 2, 3, 4, or 5 days, optionally 3 days, and/or the second period of time is at least 10, 11, 12, 13, 14, 15, 16, 17, 18, 19, or 20 days.

82. The method of any one of embodiments 68-81, wherein the mid/hindgut spheroids comprise a CD31+ vascular network surrounding the CDX2+/FOXA2+ mid/hindgut epithelium.

83. The method of any one of embodiments 68-82, wherein the mid/hindgut spheroids are the mid/hindgut spheroids of embodiment 37.

84. The vHIO produced by the method of any one of embodiments 68-75, optionally comprising a FOXF1 mutation.

85. The vHCO produced by the method of any one of embodiments 76-83, optionally comprising a FOXF1 mutation.

86. Any of the preceding embodiments, wherein the method is a method disclosed in Example 8.

### BRIEF DESCRIPTION OF THE DRAWINGS

In addition to the features described herein, additional features and variations will be readily apparent from the following descriptions of the drawings and exemplary embodiments. It is to be understood that these drawings depict embodiments and are not intended to be limiting in scope.

FIG. 1A-B depict an embodiment of single cell RNA-seq analysis of EC from 15 human fetal organs. FIG. 1A depicts a UMAP projection of ECs from different organs. ECs (total of 89,897 cells) were first selected based on CDH5 expression in each organ. FIG. 1B depicts a heatmap of the highly enriched genes in ECs from each organ.

FIG. 2A-B depict an embodiment of balancing endoderm and mesoderm co-differentiation from human iPSCs. FIG. 2A depicts a schematic illustration of a differentiation protocol for endoderm-mesoderm co-development. FIG. 2B depicts immunofluorescent staining of the spheroids on day 3. CHIR99021: 12 μM. Scale bar: 50 μm.

FIG. 3A-C depict an embodiment of vascularization and anterior-posterior (A-P) patterning of the gut tube. FIG. 3A depicts a schematic illustration of a differentiation protocol for the co-development of the vasculature and the gut tube. FIG. 3B depicts immunostaining of the foregut (left panel) versus mid/hindgut (right panel) spheroids on day 7. FIG. 3C depicts immunostaining of the vasculature (CD31) in foregut (left panel) and mid/hindgut (right panel) spheroids.

FIG. 4A-D depict an embodiment of vascularization of distal lung organoids. FIG. 4A depicts a schematic illustration of a differentiation protocol for the co-development of vasculature and distal lung epithelial progenitors. FIG. 4B depicts immunostaining of markers for distal lung epithelial progenitors on day 17. FIG. 4C depicts immunostaining of a vascularized distal lung organoid on day 17, showing the presence of CD31 vasculature. FIG. 4D depicts a brightfield view of a day 20 human vascularized distal lung organoid. Scale bar: 200 μm.

FIG. 5A-B depicts an embodiment of differences in properties and development of proximal and distal lung. FIG. 5A depicts a schematic of branching lung depicting key proximodistal patterning markers and regulators. FIG. 5B depicts maturation markers specific for distal and proximal lung tissue.

FIG. 6A-B depicts an embodiment of cell-cell interactions during human lung embodiment. CellChat analysis was performed based on a human fetal lung atlas. FIG. 6A depicts a global view of the inferred cell-cell communications in developing fetal human lung. Node size is proportional to the number of cells. Edge thickness is proportional to the number of inferred interactions. FIG. 6B depicts ligand-receptor signaling from diverse lung cells to the vascular ECs. Node size is proportional to the statistical significance of a ligand-receptor interaction in a sender-receiver cell pair. Interactions with statistical significance of p<0.05 were shown.

FIG. 7 depicts an embodiment of a schematic illustration of an in vitro perfusion system.

FIG. 8 depicts an embodiment of qPCR results of EC marker genes in control vs. FOXF1 mutant iPSC-derived ECs or vessel organoid (VO). N=3 technical repeats.

FIGS. 9A-J depict an embodiment of vascularization of small intestinal and colonic organoids. FIG. 9A depicts a schematic illustration of a differentiation protocol for the co-development of vasculature and small intestinal organoids. Immunostaining of markers for posterior gut tube (FIG. 9B), intestinal organoid (FIG. 9C), and vascular ECs (FIG. 9D) on day 30. Human colonic organoids (HCOs) were used as negative controls for GATA4 staining. FIG. 9F depicts a schematic illustration for a differentiation protocol for the co-development of vasculature and colonic organoids. Immunostaining of markers for posterior gut tube (FIG. 9G), colonic organoid (FIG. 9H), and vascular ECs (FIG. 9I) on day 30. Human intestinal organoids (HIOs) were used as a negative control for SATB2 staining. Scale bar: 50 μm.

FIG. 10 depicts an embodiment of a schematic for differentiation of various vascularized organoids.

FIG. 11 depicts embodiments of exemplary growth and differentiation culture media that may be used for the methods described herein.

FIG. 12 depicts an embodiment of an overview of methods to differentiate human pluripotency stem cells (hPSCs) into vascularized distal lung organoids, vascularized human intestinal organoids, and vascularized colonic organoids.

FIG. 13 depicts an embodiment of generating vascularized lung organoids from foregut derivatives and an embodiment of generating vascularized intestinal organoids and vascularized colonic organoids from midgut/hindgut derivatives.

FIG. 14A-C depict embodiments of the impact of CHIR99201 and BMP4 on mesoderm/endoderm ratio.

FIG. 15A-C depicts an embodiment of vascularized lung organoids expressing human lung EC specific markers. FIG. 15A depicts human lung specific markers from human cell atlas maps. FIG. 15B depicts human fetal tissue immunostained with markers including human lung embryonic cell marker HPGD (green). FIG. 15C depicts immunostained vascularized lung organoids and intestinal organoids, and shows that human lung embryonic cell marker HPGD (green) is exclusively expressed by vascularized lung organoids and not intestinal organoids.

FIG. 16A-E depicts an embodiment of a characterization of Day 3 meso/endoderm organoids by single-cell RNA-seq. FIG. 16A depicts embryoid bodies (EBs) receiving various duration of BMP4 stimulation (0, 1, 2, and 3 days) were subjected to single-cell RNA-seq at day 3. FIG. 16B depicts a feature plot of cell distributions under different BMP4 treatment durations. FIG. 16C depicts clustering of d3 meso/endoderm based on marker genes.

FIG. 16D depicts a reference map comparison of d3 meso/endoderm to CS7 gastrulation human embryo (AM: Advanced mesoderm; EM: Emergent mesoderm; PS: Primitive Streak). FIG. 16E depicts a cluster projection of day 3 meso/endoderm organoids to mouse early gastrulation embryos. i. UMAP of mouse E6.5˜E8.5 embryos. ii. Reverse projection of day 3 organoids to mouse embryos map. Accumulated stimulation by BMP4 augmented mesenchymal population, while reduced neural lineage (e.ii).

FIG. 17A-D depicts an embodiment of a characterization of Day 7 vascularized foregut and mid/hindgut organoids by single-cell RNA-seq. FIG. 17A depicts representative UMAP projection of foregut organoid (1-day treatment of BMP4 from day 1˜3) and mid/hindgut organoid (3-days treatment of BMP4 from day 1-3). FIG. 17B-D depict reverse projection of human vascularized gut tube organoids to mouse E8.75 gut tube atlas. FIG. 17B depicts mouse E8.75 embryonic gut tube containing both anterior (A) and posterior (P) portion. Gut tube (endoderm), mesenchyme, and endothelium populations are segregated into A-P subclusters. FIG. 17C depicts Day 7 foregut organoid reverse projection to mouse E8.75 gut tube. Compared to no BMP4, treatment with one-day BMP4 generated purer anterior gut tube endoderm and more anterior endothelium. FIG. 17D depicts Day 7 mid/hindgut organoid reverse projection to mouse E8.75 tube. Longer treatment of BMP4 during the first three-day differentiation induced more posterior gut tube endoderm, mesenchyme, and endothelium.

FIG. 18A-B depicts an embodiment of abnormal phenotypes of vHLuO and vHIO from FOXF1-mutant iPSC-lines accessed by immunofluorescence staining. FIG. 18A depicts compared to normal control (n=2), D31 distal vHLuO from FOXF1-mutant iPSC (n=2) showed mixed lung (TTF1+) and posterior gut tube (CDX2+) in epithelial structure (FOXA2+). Besides, FOXF1-distal vHLuO lost distal lung marker SOX9 expression. FIG. 18B depicts compared to normal control (n=2), D31 vHIO from FOXF1-mutant iPSCs (n=2) loss posterior gut tube marker (CDX2) with mixed anterior gut tube population (SOX2). Mesenchymal marker FOXF1 was also significantly suppressed. No clear expression difference of intestinal maker GATA4 or endothelium (CD31). Colonic marker SATB2 was slightly elevated in FOXF1-mutant vHIO.

### DETAILED DESCRIPTION

Human organogenesis is a highly orchestrated process that requires coordination between cells derived from different germ layers. Lateral plate mesoderm (LPM)-derived splanchnic mesoderm (SM) forms the outer layer of the primitive gut tube. The proper arrangement of splanchnic mesoderm provides the appropriate signals pivotal for the normal development of the embryonic gut tube and its derivatives. In tandem, the vasculature co-develops and intertwines with splanchnic mesoderm to support gut tube development, while reciprocally receiving molecular cues to adopt organ-specific fingerprints. This early developmental process is challenging to capture in human samples at early gestation stages.

The inter-lineage crosstalk between the endodermal and mesodermal cells is important for maintaining proper morphogenesis during early development. However, to date, there is no experimentally tractable system that contains both lineages in a reliable manner to study their mutual interaction during human organogenesis. While prior work mainly focused on single germ layer differentiation, it is noted that endoderm and mesoderm specification shared similar regulatory networks: the formation of both germ layers is facilitated by the inhibition of insulin and phosphoinositide 3-kinase signaling, and can be induced by a similar set of growth factors including Wnt, BMP, and TGF-b, although the quantitative combination of these signaling pathways vary for endoderm and mesoderm diversification. Based on these developmental cues, disclosed herein in some embodiments are novel methods using pluripotent stem cells to simultaneously induce both mesoderm-derived ECs/mesenchyme and endoderm-derived lung or intestinal epithelium within a single 3D organoid system by balancing Wnt, BMP, and TGF-b activation. Different from prior protocols for differentiating cells arising from a single germ layer, the establishment of the new methods disclosed herein involved extensive testing of various combinations of small molecules for maintaining both germ layers, given the additional intrinsic signal provided by the mesoderm-derived cells. The intimate co-development of the EC-epithelium-mesenchyme allows for the study of direct signaling transduction among multiple cell types throughout the development process in an organ-specific manner.

Different types of iPSC derived organoid models have previously been established to re-create the architecture and physiology of their in vivo counterparts with remarkable detail. Recent progress in generating endodermal organoids has been made towards improving the maturation and patterning of the epithelial cells. Although it is recognized that human vasculature co-develops with other cell types during organoid formation to provide critical growth factors and improve regional patterning, to date, there is no organoid model that contains a vascular network co-developed with other organ-specific cell types from the stem cell stage. To vascularize organoids, recent efforts have focused on techniques ranging from incorporating terminally differentiated human umbilical vein endothelial cells (HUVECs) to the organoid, expanding the endogenous endothelial progenitor cells with VEGF, in vivo transplantation, “organ-on-a-chip” engineering approaches, and introducing mechanical stimuli or inducible genetic circuits. Despite various levels of success have been achieved with brain, liver, and kidney organoids, these methods were not effective to vascularize most of the endodermal organs, especially in the lung, and did not recapitulate the cell type diversity, spatial organization, and physiologically relevant microenvironment that lets the stem cells grow into tissue. As disclosed herein, instead of assembling different cell types and organoids in an artificial manner, novel approaches to vascularize these organoids via a co-differentiation strategy using iPSCs (e.g. human iPSCs), which enables intimate cell-cell communication from the very beginning of organogenesis, are provided. Herein, we devised an optimized method (see, e.g., Example 8) where embryonic bodies are exposed to continual stimulation of Nodal signals and pulsatile induction by several other morphogens, such as Wnt, FGF2, and BMP4, essentially mimicking the early gastrulation stage. During later organogenesis phases, regionalization of primitive gut tube towards different organs is accomplished by manipulation of germ layer composition in primordial development era and support from appropriate vascular and mesenchymal compartments. Organoids with functional vasculature may exhibit more mature structural organization and functionality in a dish, and can serve as a better source for cell replacement therapy and organ regeneration. This innovative co-differentiation strategy has an extraordinary impact on the broad scientific community, as it can serve as a human iPSC derived platform to study the early developmental events in various organ systems including but not limited to those provided herein.

Herein, we demonstrate that in embodiments of the vascularized lung organoids (vLuO) provided herein, ECs co-developed with the lung epithelium adopted lung EC genetic signatures. Interestingly, in addition to the environmental factors, ECs at different parts of the organ may also have different origins. For example, a multipotent cardiopulmonary progenitor only gives rise to the ECs populating the proximal but not distal lung. Because of the lack of a modeling system during human development, it was unclear how EC specification occurs in response to both intrinsic and extrinsic factors that drive the acquisition of their unique features. Herein, ECs were differentiated from the iPSC stage and interacted directly with the other cell types in the organoid to gain organ-specific features over time.

Given that in some embodiments the endodermal organoids provided herein comprise a vascular network being co-developed with other cell types, they can serve as a powerful tool for the study of the role of vascular deficiency in, for example, pulmonary and gastrointestinal anomalies. For example, in some embodiments iPSCs can be obtained from patients with a mutation in FOXF1, which is a gene that specifically labels splanchnic mesoderm and remains highly expressed in mesoderm-derived ECs. As FOXF1 mutant patients exhibit abnormalities in both lung vasculature and intestine, the impact of the FOXF1 mutation on vascular development itself, and the mechanism by which FOXF1 mutant ECs affect the formation and functions of the lung and intestinal organoids is assessed.

Moving beyond the limitations of two dimensional (2D) culture with a single cell type, or the “organ-on-a-chip” model with restricted capacity for high-throughput production, embodiments of the three dimensional (3D) vascularized organoids disclosed herein allow for massive expansion and manufacturing for personalized drug screening targeting the abnormal growth of the vascular network, as well as cell replacement therapy.

In contrast to prior models that focus on making vascularized organoids by incorporating terminally differentiated ECs or “reset” ECs at a later developmental stage, the methods disclosed herein (e.g., Example 8) capture the early events during endoderm and mesoderm co-development, as well as the intimate crosstalk between the vascular progenitors and the primitive gut tube in a single spheroid, which precisely recapitulates the entire process of human organogenesis, as the vascular lineages emerge early during development. The co-development methods disclosed herein provided a physiologically relevant microenvironment to drive the patterning of the vasculature, mesenchyme, and the epithelium layer throughout the differentiation process. Using this approach, two main barriers for generating lung organoids can be overcome in some embodiments disclosed herein: lack of the vasculature, and lack of AT1 cells, which form 95% of the pulmonary gas exchange surface. These aims could not be achieved using prior organoid models as the vasculature was either not human-specific (vascularized by in vivo transplantation in mice) or not co-developed with other cell types in the organoids to capture the early events during the developmental process.

### Terms

In the following detailed description, reference is made to the accompanying drawings, which form a part hereof. Tn the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The disclosure herein uses affirmative language to describe the numerous embodiments. The disclosure also includes embodiments in which subject matter is excluded, in full or in part, such as substances or materials, method steps and conditions, protocols, or procedures.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20% 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%0, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, s-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present disclosure, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof. Other methods of producing induced pluripotent stem cells as conventionally known in the art are also envisioned.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), epiblast stem cells (EpiSC), and induced pluripotent stem cells.

In developmental biology, cellular differentiation is the process by which a less specialized cell becomes a more specialized cell type. As used herein, the term “differentiation” or “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of target stem cells.

### Cell Differentiation

In some embodiments, known methods for producing downstream cell types from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells or induced pluripotent stem cells. Embryonic stem cells can be derived from the embryonic inner cell mass or from the embryonic gonadal ridges. Embryonic stem cells or germ cells can originate from a variety of animal species including, but not limited to, various mammalian species including humans.

In some embodiments, human embryonic stem cells are used to produce definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof. In some embodiments, iPSCs are used to produce definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof. In some embodiments, human iPSCs (hiPSCs) are used to produce definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof.

In some embodiments, the pluripotent stem cells are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the pluripotent stem cells are cultured in growth media that supports the growth of stem cells. In some embodiments, the pluripotent stem cells are cultured in stem cell growth media. In some embodiments, the stem cell growth media is RPMI 1640, DMEM, DMEM/F12, or Advanced DMEM/F12. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

Tn some embodiments, pluripotent stem cells are prepared from somatic cells. In some embodiments, pluripotent stem cells are prepared from biological tissue obtained from a biopsy. In some embodiments, the pluripotent stem cells are cryopreserved. In some embodiments, the somatic cells are cryopreserved. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate.

In some embodiments, iPSCs are expanded in cell culture. In some embodiments, iPSCs are expanded in Matrigel. In some embodiments, the iPSCs are expanded in cell culture comprising a ROCK inhibitor (e.g. Y-27632).

In some embodiments, proteins, activators, or inhibitors of the FGF, Wnt, BMP, TGF-b, or retinoic acid (RA) pathways, or any combination thereof, are used to mimic development in culture to obtain various cell types used herein that are differentiated from pluripotent stem cells. In some embodiments, cellular constituents associated with the FGF, Wnt, BMP, TGF-b or RA signaling pathways, for example, natural inhibitors, antagonists, activators, or agonists of the pathways can be used to result in inhibition or activation of the FGF, Wnt, BMP, TGF-b or RA signaling pathways. In some embodiments, siRNA and/or shRNA targeting cellular constituents associated with the FGF, Wnt, BMP, TGF-b or retinoic acid signaling pathways are used to inhibit or activate these pathways. Furthermore, the methods disclosed herein may also involve the use of an EGF pathway activator acting as a mitogen, which promotes proliferation and growth of desired cell populations.

In some embodiments, pluripotent stem cells, definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof, are contacted with a Wnt pathway activator or Wnt pathway inhibitor. In some embodiments, the Wnt pathway activator comprises a Wnt protein. In some embodiments, the Wnt protein comprises a recombinant Wnt protein. In some embodiments, the Wnt pathway activator comprises Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, or any combination thereof. In some embodiments, the Wnt pathway activator comprises a GSK3 pathway inhibitor. In some embodiments, the Wnt pathway activator comprises CHIR99021, CHIR 98014, AZD2858, BIO, AR-A014418, SB 216763, SB 415286, aloisine, indirubin, alsterpaullone, kenpaullone, lithium chloride, TDZD 8, or TWS119, or any combination thereof. In some embodiments, the Wnt pathway inhibitor comprises C59, PNU 74654, KY-02111, PRI-724, FH-535, DIF-1, or XAV939, or any combination thereof. In some embodiments, the cells are not treated with a Wnt pathway activator or Wnt pathway inhibitor. The Wnt pathway activator or Wnt pathway inhibitor provided herein may be used in combination with any of the other growth factors, pathway activators, or pathway inhibitors provided herein.

In some embodiments, pluripotent stem cells, definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof, are contacted with an FGF pathway activator. In some embodiments, the FGF pathway activator comprises an FGF protein. In some embodiments, the FGF protein comprises a recombinant FGF protein. In some embodiments, the FGF pathway activator comprises one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, or FGF23. In some embodiments, the cells are not treated with an FGF pathway activator. The FGF pathway activator provided herein may be used in combination with any of the other growth factors, pathway activators, or pathway inhibitors provided herein.

In some embodiments, pluripotent stem cells, definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof, are contacted with a BMP pathway activator or BMP pathway inhibitor. In some embodiments, the BMP pathway activator comprises a BMP protein. In some embodiments, the BMP protein is a recombinant BMP protein. In some embodiments, the BMP pathway activator comprises BMP1, BMP2, BMP3, BMP4, BMP5, BMP6, BMP7, BMP8a, BMP8b, BMP10, BMP11, BMP15, IDE1, or IDE2, or any combination thereof. In some embodiments, the BMP pathway inhibitor comprises Noggin, Dorsomorphin, RepSox, LY364947, LDN193189, SB431542, or any combination thereof. In some embodiments, the cells are not treated with a BMP pathway activator or BMP pathway inhibitor. The BMP pathway activator or BMP pathway inhibitor provided herein may be used in combination with any of the other growth factors, pathway activators, or pathway inhibitors provided herein.

In some embodiments, pluripotent stem cells, definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof, are contacted with a retinoic acid pathway activator. In some embodiments, the retinoic acid pathway activator comprises retinoic acid, all-trans retinoic acid, 9-cis retinoic acid, CD437, EC23, BS 493, TTNPB, or AM580, or any combination thereof. In some embodiments, the cells are not treated with a retinoic acid pathway activator. The retinoic acid pathway activator provided herein may be used in combination with any of the other growth factors, pathway activators, or pathway inhibitors provided herein.

In some embodiments, pluripotent stem cells, definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof, are contacted with an EGF pathway activator. In some embodiments, the EGF pathway activator is EGF. In some embodiments, the cells are not treated with an EGF pathway activator. The EGF pathway activator provided herein may be used in combination with any of the other growth factors, pathway activators, or pathway inhibitors provided herein.

In some embodiments, pluripotent stem cells, definitive endoderm, lateral plate mesoderm, mesendoderm spheroids, foregut spheroids, mid/hindgut spheroids, ventral anterior foregut spheroids, vascularized distal lung organoids, vascularized proximal lung organoids, vascularized small intestine organoids, vascularized colonic organoids, or any combination thereof, are contacted with a TGF-beta (TGF-b) pathway activator or TGF-b pathway inhibitor. In some embodiments, the TGF-b family comprises bone morphogenetic protein (BMP), growth and differentiation factor (GDF), anti-Müllerian hormone, Activin, and Nodal pathways. In some embodiments, the TGF-b pathway activator comprises TGF-b 1, TGF-b 2, TGF-b 3, Activin A, Activin B, Nodal, a BMP, IDE1, IDE2, or any combination thereof. Tn some embodiments, the TGF-b pathway inhibitor comprises A8301, RepSox, LY365947, SB431542, or any combination thereof. In some embodiments, the cells are not treated with a TGF-b pathway activator or TGF-b pathway inhibitor. The TGF-b pathway activator or TGF-b pathway inhibitor provided herein may be used in combination with any of the other growth factors, pathway activators, or pathway inhibitors provided herein.

In some embodiments, for any of the small molecule compounds, pathway activators, pathway inhibitors, or growth factors, the cells are contacted for a time that is, is about, is at least, is at least about, is not more than, or is not more than about, 1 hour, 2 hours, 3 hours, 4 hours, 5 hours, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 1 hour to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the PSCs are differentiated into definitive endoderm cells. In some embodiments, the PSCs are differentiated into lateral plate mesoderm. In some embodiments, the PSCs are differentiated into mesendoderm spheroids. In some embodiments, the PSCs are differentiated into foregut spheroids. In some embodiments, the PSCs are differentiated into mid/hindgut spheroids. In some embodiments, the PSCs are differentiated into ventral anterior foregut spheroids. In some embodiments, the PSCs are differentiated into vascularized distal lung organoids. In some embodiments, the PSCs are differentiated into vascularized proximal lung organoids. In some embodiments, the PSCs are differentiated into vascularized small intestine organoids. In some embodiments, the PSCs are differentiated into vascularized colonic organoids.

In some embodiments, any of the cells disclosed herein may be cryopreserved for later use. In some embodiments, the cells are cryopreserved according to methods generally known in the art.

### Methods of Making Mesendoderm and Vascularized Lung Organoids

Generating various types of organoids from pluripotent stem cells, and precursors thereof, such as definitive endoderm, gut endoderm, foregut endoderm, or mid/hindgut endoderm, are generally known in the art. Exemplary methods may be found in PCT publications WO 2011/140441, WO 2015/183920, WO 2016/061464, WO 2017/192997, WO 2018/106628, WO 2018/200481, WO 2019/074793, WO 2020/160371, WO 2021/030373, and WO 2020/243633, each of which is hereby expressly incorporated by reference in its entirety.

Methods of producing mesoderm, for example, lateral plate mesoderm, from pluripotent stem cells can be found in PCT publication WO 2021/041443, which is hereby expressly incorporated by reference in its entirety.

Lung organoids can be produced by some methods generally known in the art. Examples of lung organoids produced from pluripotent stem cells can be found in PCT publication WO 2019/074793, U.S. publications 2016/0312191, 2018/0344901, and 2020/0149004, and Gotoh et al. “Generation of Alveolar Epithelial Spheroids via Isolated Progenitor Cells from Human Pluripotent Stem cells” Stem Cell Reports (2014) 3(3):394-403; Dye et al. “In vitro generation of human pluripotent stem cell derived lung organoids” eLife 4:e05098; Hawkins et al. “Prospective isolation of NKX2-1-expressing human lung progenitors derived from pluripotent stem cells” J. Clin. Invest. (2017) 127(6):2277-2294; McCauley et al. “Efficient Derivation of Functional Human Airway Epithelium from Pluripotent Stem Cells via Temporal Regulation of Wnt Signaling” Cell Stem Cell (2017) 20(6):844-857; Chen et al. “Three-dimensional model of human lung development and disease from pluripotent stem cells” Nature Cell Biology (2017) 19:542-549; and Miller et al. “Generation of lung organoids from human pluripotent stem cells in vitro” Nat. Protoc. (2019) 14(2):518-540, each of which is hereby expressly incorporated by reference in its entirety.

In some embodiments, the organoids, for example lung organoids, produced according to some of these methods in the art may benefit from proceeding from mesendoderm spheroids disclosed herein comprising both definitive endoderm and lateral plate mesoderm, for example, to enhance vascularization of the organoid without the need for ectopic transplantation.

Small intestine (intestinal) organoids can be produced by some methods generally known in the art. Examples of intestinal organoids produced from pluripotent stem cells can be found in PCT publications WO 2011/140441, WO 2016/061464, WO 2018/200481, WO 2020/160371, and WO 2021/030373, each of which is hereby expressly incorporated by reference in its entirety. In some embodiments, the intestinal organoids produced according to some of these methods in the art may benefit from proceeding from mesendoderm spheroids disclosed herein comprising both definitive endoderm and lateral plate mesoderm, for example, to enhance vascularization of the organoid without the need for ectopic transplantation.

Large intestine (colonic) organoids can be produced by some methods generally known in the art. Examples of colonic organoids produced from pluripotent stem cells can be found in PCT publication WO 2018/106628, which is hereby expressly incorporated by reference in its entirety. In some embodiments, the colonic organoids produced according to some of these methods in the art may benefit from proceeding from mesendoderm spheroids disclosed herein comprising both definitive endoderm and lateral plate mesoderm, for example, to enhance vascularization of the organoid without the need for ectopic transplantation.

Some embodiments described herein are methods of preparing organoids using mesendoderm populations, such as mesendoderm spheroids. These mesendoderm populations or mesendoderm spheroids comprise both definitive endoderm and lateral plate mesoderm. Organoids differentiated from these mesendoderm spheroids have robust amounts of both epithelium and mesenchyme, and also exhibit vascularization, which is a feature not seen in previous organoids through normal differentiation processes and was only accomplished by exogenous mixing or ectopic transplantation into an animal. Methods of producing these mesendoderm spheroids from pluripotent stem cells are also disclosed herein.

Some embodiments described herein are methods of producing mesendoderm spheroids comprising both definitive endoderm and lateral plate mesoderm. In some embodiments, the method comprises one or more of the methods disclosed in Example 8. In some embodiments, the methods comprise a) contacting pluripotent stem cells with a TGF-b pathway activator, a BMP pathway activator, and a Wnt pathway activator; and b) contacting the cells of step a) with a TGF-b pathway activator and a BMP pathway activator, without a Wnt pathway activator, thereby differentiating the pluripotent stem cells to mesendoderm spheroids comprising both FOXF2+ definitive endoderm and HAND1+ lateral plate mesoderm. In some embodiments, the FOXA2+ definitive endoderm and the HAND1+ lateral plate mesoderm are approximately at a 1:2, 1:1.5, 1:1, 1.5:1, or 2:1 ratio. In some embodiments, the FOXA2+ definitive endoderm and the HAND1+ lateral plate mesoderm are approximately at a 1:1 ratio. In some embodiments, the TGF-b pathway activators and/or BMP pathway activators of steps a) and b) are not necessarily the same. In some embodiments, the TGF-b pathway activators and/or BMP pathway activators of steps a) and b) are the same. In some embodiments, the cells of step a) and/or b) are further contacted with a serum supplement, such as fetal bovine serum (FBS), or an FBS substitute. In some embodiments, the serum supplement is at a concentration that is, is about, is at least, is at least about, is not more than, or is not more than about, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%, or a range defined by any two of the preceding values. In some embodiments, the cells of step a) and/or b) are contacted with the serum supplement such that the concentration of the serum supplement during step b) is higher than the concentration during step a). In some embodiments, the lateral plate mesoderm surrounds the definitive endoderm in the mesendoderm spheroids. In some embodiments, step a) is performed for an amount of time that is, is about, is at least, is at least about, is not more than, or is not more than about, 24 hours to about 48 hours, for example, 24, 25, 26, 27, 28, 29, 30, 31, 32, 33, 34, 35, 36, 37, 38, 38, 39, 40, 41, 42, 43, 44, 45, 46, 47, or 48 hours, or a range defined by any two of the preceding values. In some embodiments, step a) is performed for about 26, 27, 28, 29, or 30 hours. In some embodiments, step a) is performed for about 28 hours. In some embodiments, step b) is performed for an amount of time that is, is about, is at least, is at least about, is not more than, or is not more than about, 24 hours to about 72 hours, for example, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours, or a range defined by any two of the preceding values. In some embodiments, step b) is performed no more than 72 hours. In some embodiments, step b) is performed for about 42, 43, 44, 45, 46, 47, 48, 49, 50, 51, 52, 53, 54, 55, 56, 57, 58, 59, or 60 hours, or a range defined by any two of the preceding values. In some embodiments, step b) is performed for about 44 hours. In some embodiments, the TGF-b pathway activator of step a) and/or b) is TGF-beta 1, TGF-beta 2, TGF-beta 3, Activin A, Activin B, Nodal, a BMP, IDE1, IDE2, or any combination thereof, optionally Activin A. In some embodiments, the TGF-b pathway activator of step a) and/or b) is provided at a concentration that is, is about, is at least, is at least about, is not more than, or is not more than about, about 50, 60, 70, 80, 90, 100, 110, 120, 130, 140, or 150 ng/mL, or any concentration within a range defined by any two of the aforementioned concentrations. In some embodiments, the TGF-b pathway activator is provided at a concentration that is, is about, is at least, is at least about, is not more than, or is not more than about, 100 ng/mL. In some embodiments, the BMP pathway activator of step a) and/or b) is BMP1, BMP2, BMP3, BMP4, BMP5, BMP6, BMP7, BMP8a, BMP8b, BMP10, BMP11, BMP15, IDE1, IDE2, or any combination thereof, optionally BMP4. In some embodiments, the BMP pathway activator of step a) and/or b) is provided at a concentration that is, is about, is at least, is at least about, is not more than, or is not more than about, about 10, 20, 30, 40, 50, 60, 70, 80, 90, or 100 ng/mL, or any concentration within a range defined by any two of the aforementioned concentrations. In some embodiments, the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, TWS119, or any combination thereof, optionally CHIR99021. In some embodiments, the Wnt pathway activator is provided at a concentration that is, is about, is at least, is at least about, is not more than, or is not more than about, about 1, 2, 3, 4, 5, 6, 7, 8, 9, 10, 11, 12, 13, 14, 15, 16, 17, 18, 19, 20, 21, 22, 23, or 24 μM, or any concentration within a range defined by any two of the aforementioned concentrations, optionally 9, 10, 11, or 12 μM. In some embodiments, prior to the contacting of step a) the pluripotent stem cells are in the form of spheroids. In some embodiments, the pluripotent stem cells are formed into spheroids by aggregation. In some embodiments, the pluripotent stem cells are formed into spheroids with the use of an Aggrewell plate (StemCell Technologies), or other suitable substitute that forms regularly sized aggregates of cell populations. In some embodiments, for the duration of steps a) and b), the pluripotent stem cells are kept in suspension, optionally with shaking, and/or not as a monolayer.

Also disclosed herein are the mesendoderm spheroids produced by any of the methods disclosed herein.

Also disclosed herein are methods of producing foregut spheroids from mesendoderm spheroids disclosed herein. In some embodiments, the method comprises one or more of the methods disclosed in Example 8. In some embodiments, the methods comprise contacting mesendoderm spheroids with a BMP pathway inhibitor, a TGF-b pathway inhibitor, and optionally a Hedgehog pathway activator, for a period of time sufficient to differentiate the mesendoderm spheroids to foregut spheroids comprising SOX2+/FOXA2+ foregut epithelium and FOXF1+ splanchnic mesoderm. In some embodiments, the period of time sufficient to differentiate the mesendoderm spheroids to foregut spheroids is, is about, is at least, is at least about, is not more than, or is not more than about, about 2 days to 6 days, such as about 2, 3, 4, 5, or 6 days, or a range defined by any two of the preceding values. In some embodiments, the BMP pathway inhibitor is Noggin, Dorsomorphin, RepSox, LY364947, LDN193189, follistatin, chordin, or any combination thereof, optionally Noggin. In some embodiments, the BMP pathway inhibitor is provided at a concentration that is, is about, is at least, is at least about, is not more than, or is not more than about, 50, 60, 70, 80, 90, 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations. In some embodiments, the TGF-b pathway inhibitor is A8301, RepSox, LY365947, SB431542, or any combination thereof, optionally SB431542. In some embodiments, the TGF-b pathway inhibitor is provided at a concentration that is, is about, is at least, is at least about, is not more than, or is not more than about, about 1, 2, 3, 4, 5, 6, 7, 8, 9, 10, 11, 12, 13, 14, 15, 16, 17, 18, 19, or 20 μM, or any concentration within a range defined by any two of the aforementioned concentrations, optionally 10 μM. In some embodiments, the Hedgehog pathway activator is Smoothened agonist (SAG). In some embodiments, the Hedgehog pathway activator is provided at a concentration of about 0.5, 1, 2, 3, 4, or 5 μM or any concentration within a range defined by any two of the aforementioned concentrations, optionally 1 μM. In some embodiments, the methods further comprise contacting the mesendoderm spheroids with vascular endothelial growth factor (VEGF) to generate a CD31+ vascular network surrounding the SOX2+/FOXA2+ foregut epithelium. In some embodiments, the VEGF is provided at a concentration that is, is about, is at least, is at least about, is not more than, or is not more than about, about 10, 20, 30, 40, 50, 60, 70, 80, 90, or 100 ng/mL, or any concentration within a range defined by any two of the aforementioned concentrations. In some embodiments, the VEGF is VEGF121 or VEGF165. In some embodiments, the methods further comprise contacting the mesendoderm spheroids with a retinoic acid pathway activator, optionally wherein the retinoic acid pathway activator is retinoic acid, all-trans retinoic acid, 9-cis retinoic acid, CD437, EC23, BS 493, TTNPB, AM580, or any combination thereof. In some embodiments, the mesendoderm spheroids are not contacted with a Wnt pathway activator and/or an FGF pathway activator. In some embodiments, the mesendoderm spheroids are not contacted with FGF2. In some embodiments, the mesendoderm spheroids are the mesendoderm spheroids produced by any one of the methods disclosed herein.

Also disclosed herein are the foregut spheroids produced by any of the methods disclosed herein.

Also disclosed herein are methods of producing mid/hindgut spheroids from mesendoderm spheroids disclosed herein. In some embodiments, the method comprises one or more of the methods disclosed in Example 8. In some embodiments, the methods comprise contacting mesendoderm spheroids with a Wnt pathway activator and an FGF pathway activator for a period of time sufficient to differentiate the mesendoderm spheroids to mid/hindgut spheroids comprising CDX2+/FOXA2+ mid/hindgut epithelium and FOXF1+ splanchnic mesoderm. In some embodiments, the period of time sufficient to differentiate the mesendoderm spheroids to mid/hindgut spheroids is, is about, is at least, is at least about, is not more than, or is not more than about, 2 days to 6 days. In some embodiments, the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, TWS119, or any combination thereof, optionally CHIR99021. In some embodiments, the Wnt pathway activator is provided at a concentration that is, is about, is at least, is at least about, is not more than, or is not more than about, about 1, 2, 3, 4, 5, 6, 7, 8, 9, or 10 μM, or any concentration within a range defined by any two of the aforementioned concentrations. In some embodiments, the FGF pathway activator is FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, FGF23, or any combination thereof, optionally FGF4. In some embodiments, the FGF pathway activator is provided at a concentration that is, is about, is at least, is at least about, is not more than, or is not more than about, about 100, 200, 300, 400, 500, 600, 700, 800, 900, or 1000 ng/mL, or any concentration within a range defined by any two of the aforementioned concentrations. In some embodiments, the methods further comprise contacting the mesendoderm spheroids with vascular endothelial growth factor (VEGF) to generate a CD31+ vascular network surrounding the CDX2+/FOXA2+ mid/hindgut epithelium. In some embodiments, the VEGF is provided at a concentration that is, is about, is at least, is at least about, is not more than, or is not more than about, about 10, 20, 30, 40, 50, 60, 70, 80, 90, or 100 ng/mL, or any concentration within a range defined by any two of the aforementioned concentrations. In some embodiments, the VEGF is VEGF121 or VEGF165. Tn some embodiments, the mesendoderm spheroids are the mesendoderm spheroids produced by any one of the methods disclosed herein.

Also disclosed herein are the mid/hindgut spheroids produced by any of the methods disclosed herein.

Also disclosed herein are methods of producing ventral anterior foregut spheroids. In some embodiments, the method comprises one or more of the methods disclosed in Example 8. In some embodiments, the methods comprise contacting any one of the foregut spheroids disclosed herein with retinoic acid for a period of time sufficient to differentiate the foregut spheroids to ventral anterior foregut spheroids. In some embodiments, the period of time sufficient to differentiate the foregut spheroids to ventral anterior foregut spheroids is, is about, is at least, is at least about, is not more than, or is not more than about, 1 day.

Also disclosed herein are the ventral anterior foregut spheroids produced by any of the methods disclosed herein.

Also disclosed herein are methods of producing vascularized distal lung organoids (vLuO). In some embodiments, the method comprises one or more of the methods disclosed in Example 8. In some embodiments, the methods comprise a) contacting ventral anterior foregut spheroids disclosed herein with a Wnt pathway activator, a BMP pathway activator, and VEGF, and optionally a retinoic acid pathway activator, for a period of time sufficient to differentiate the ventral anterior foregut spheroids into lung progenitors, and b) contacting the lung progenitors with a distal lung specification medium comprising a Wnt pathway activator, one or more FGF pathway activators, and VEGF, for a period of time sufficient to differentiate the lung progenitors to vLuO, wherein the vLuO comprises TTF1/NKX2-1+ distal lung epithelial progenitors, is positive for SOX9, exhibits branching morphogenesis, and receives FGF10 signaling secreted by distal mesenchyme derived from FOXF1+ splanchnic mesoderm. In some embodiments, the ventral anterior foregut spheroids comprise a CD31+ vascular network surrounding the SOX2+/FOXA2+ foregut epithelium. In some embodiments, the ventral anterior foregut spheroids are the ventral anterior foregut spheroids produced by any of the methods disclosed herein. In some embodiments, the ventral anterior foregut spheroids of step a) are embedded in a basement membrane matrix and are contacted in a stationary culture for a first period of time and in a culture with shaking for a second period of time. In some embodiments, the shaking for the second period of time promotes circulation of nutrition and promotes vascularization. In some embodiments, the first period of time is, is about, is at least, is at least about, is not more than, or is not more than about, 1, 2, 3, 4, or 5 days, optionally 3 days. In some embodiments, the first period of time is at least 3, 4, or 5 days, optionally at least 3 days. In some embodiments, the second period of time is, is about, is at least, is at least about, is not more than, or is not more than about, 5, 6, 7, 8, 9, or 10 days, optionally 7 days. In some embodiments, the second period of time is at least 5, 6, 7, 8, 9, or 10 days, optionally at least 5 days. In some embodiments, the distal lung specification medium further comprises dexamethasone, cAMP, and 3-isobutyl-1-methylxanthine (IBMX). In some embodiments, the Wnt pathway activator of step a) and/or step b) is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, TWS119, or any combination thereof, optionally CHIR99021. In some embodiments, the Wnt pathway activator of step a) and/or step b) is provided at a concentration that is, is about, is at least, is at least about, is not more than, or is not more than about, about 1, 2, 3, 4, 5, 6, 7, 8, 9, or 10 μM, or any concentration within a range defined by any two of the aforementioned concentrations, optionally 3 μM. In some embodiments, the one or more FGF pathway activators are selected from FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, FGF23, or any combination thereof. In some embodiments, the one or more FGF pathway activators comprise FGF7 and FGF10. In some embodiments, the one or more FGF pathway activators consist of FGF7 and FGF10. In some embodiments, the one or more FGF pathway activators are each provided at a concentration that is, is about, is at least, is at least about, is not more than, or is not more than about, 5, 6, 7, 8, 9, 10, 11, 12, 13, 14, or 15 ng/mL, or any concentration within a range defined by any two of the aforementioned concentrations, optionally 10 ng/mL. In some embodiments, the BMP pathway activator is BMP1, BMP2, BMP3, BMP4, BMP5, BMP6, BMP7, BMP8a, BMP8b, BMP10, BMP11, BMP15, IDE1, IDE2, or any combination thereof, optionally BMP4. In some embodiments, the BMP pathway activator is provided at a concentration that is, is about, is at least, is at least about, is not more than, or is not more than about, 10, 20, 30, 40, 50, 60, 70, 80, 90, or 100 ng/mL, or any concentration within a range defined by any two of the aforementioned concentrations. In some embodiments, the retinoic acid pathway activator is retinoic acid, all-trans retinoic acid, 9-cis retinoic acid, CD437, EC23, BS 493, TTNPB, AM580, or any combination thereof, optionally all-trans retinoic acid (ATRA). In some embodiments, the VEGF is provided at a concentration that is, is about, is at least, is at least about, is not more than, or is not more than about, 10, 20, 30, 40, 50, 60, 70, 80, 90, or 100 ng/mL, or any concentration within a range defined by any two of the aforementioned concentrations. In some embodiments, the VEGF is VEGF121 or VEGF165. In some embodiments, the dexamethasone is provided at a concentration that is, is about, is at least, is at least about, is not more than, or is not more than about, 10, 20, 30, 40, 50, 60, 70, 80, 90, or 100 nM, or any concentration within a range defined by any two of the aforementioned concentrations, optionally about 50 nM. In some embodiments, the cAMP is provided at a concentration that is, is about, is at least, is at least about, is not more than, or is not more than about, 50, 100, 150, 200, 250, 300, 350, 400, 450, or 500 μM, or any concentration within a range defined by any two of the aforementioned concentrations, optionally about 100 μM. In some embodiments, the IBMX is provided at a concentration that is, is about, is at least, is at least about, is not more than, or is not more than about, 50, 100, 150, 200, 250, 300, 350, 400, 450, or 500 μM, or any concentration within a range defined by any two of the aforementioned concentrations, optionally about 100 μM. In some embodiments, the vLuO are embedded in a lung-specific extracellular matrix, optionally wherein the lung-specific extracellular matrix is isolated from human lung tissue. In some embodiments, the methods further comprise contacting the ventral anterior foregut spheroids with midkine (MDK), semaphorin-3C (SEMA3C), growth/differentiation factor-15 (GDF15), or any combination thereof.

Also disclosed herein are the vLuO produced by any of the methods disclosed herein. In some embodiments, the vLuO is positive for one or more human lung endothelial cell (EC) markers. In some embodiments the human lung EC marker is selected from the group consisting of FENDRR, NCKAP5, HPGD, KIT, and PDE3B. In some embodiments, the vLuO comprise a FOXF1 mutation. In some embodiments the FOXF1 mutation is present in the stem cells, mesendoderm spheroid, foregut spheroid and/or ventral anterior foregut spheroid from which the vLuO is derived. In some embodiments, the vLuO comprising the FOXF1 mutation can be used as a disease model for alveolar capillary dysplasia (ACD), misalignment of pulmonary vein (MPV), and/or pulmonary lymphangiectasia. In some embodiments, the vLuO are embedded in a lung-specific extracellular matrix, optionally wherein the lung-specific extracellular matrix is isolated from human lung tissue.

Also disclosed herein are methods of producing vascularized proximal lung organoids. In some embodiments, the method comprises one or more of the methods disclosed in Example 8. In some embodiments, the methods comprise a) contacting ventral anterior foregut spheroids disclosed herein with a Wnt pathway activator, a BMP pathway activator, and VEGF, and optionally a retinoic acid pathway activator, for a period of time sufficient to differentiate the ventral anterior foregut spheroids into lung progenitors, and b) contacting the lung progenitors with a proximal lung specification medium comprising one or more FGF pathway activators and VEGF for a period of time sufficient to differentiate the lung progenitors to vascularized proximal lung organoids. In some embodiments, the one or more FGF pathway activators are provided at a concentration that is greater than that used to produce distal lung organoids. In some embodiments, the ventral anterior foregut spheroids comprise a CD31+ vascular network surrounding the SOX2+/FOXA2+ foregut epithelium. In some embodiments, the ventral anterior foregut spheroids are the ventral anterior foregut spheroids produced by any of the methods disclosed herein. In some embodiments, the one or more FGF pathway activators are selected from FGF1, FGF2, FGF3, FGF4, FGF4, FGF5, FGF6, FGF7, FGF8, FGF8, FGF9, FGF10, FGF11, FGF12, FGF13, FGF14, FGF15, FGF16, FGF17, FGF18, FGF19, FGF20, FGF21, FGF22, FGF23, or any combination thereof. In some embodiments, the one or more FGF pathway activators comprise FGF2 and FGF10. In some embodiments, the one or more FGF pathway activators consist of FGF2 and FGF10. In some embodiments, the one or more FGF pathway activators are each provided at a concentration that is, is about, is at least, is at least about, is not more than, or is not more than about, 100, 110, 120, 130, 140, 150, 160, 170, 180, 190, 200, 210, 220, 230, 240, 250, 260, 270, 280, 290, or 300 ng/mL, or any concentration within a range defined by any two of the aforementioned concentrations. In some embodiments, the proximal lung specification medium further comprises dexamethasone, cAMP, and IBMX. In some embodiments, the VEGF is provided at a concentration that is, is about, is at least, is at least about, is not more than, or is not more than about, 10, 20, 30, 40, 50, 60, 70, 80, 90, or 100 ng/mL, or any concentration within a range defined by any two of the aforementioned concentrations. In some embodiments, the VEGF is VEGF121 or VEGF165. In some embodiments, the dexamethasone is provided at a concentration that is, is about, is at least, is at least about, is not more than, or is not more than about, 10, 20, 30, 40, 50, 60, 70, 80, 90, or 100 nM, or any concentration within a range defined by any two of the aforementioned concentrations, optionally about 50 nM. In some embodiments, the cAMP is provided at a concentration that is, is about, is at least, is at least about, is not more than, or is not more than about, 50, 100, 150, 200, 250, 300, 350, 400, 450, or 500 PM, or any concentration within a range defined by any two of the aforementioned concentrations, optionally about 100 μM. In some embodiments, the IBMX is provided at a concentration that is, is about, is at least, is at least about, is not more than, or is not more than about, 50, 100, 150, 200, 250, 300, 350, 400, 450, or 500 μM, or any concentration within a range defined by any two of the aforementioned concentrations, optionally about 100 μM. In some embodiments, the ventral anterior foregut spheroids are not contacted with a Wnt pathway activator. In some embodiments, the ventral anterior foregut spheroids are embedded in a lung-specific extracellular matrix during the contacting steps, optionally wherein the lung-specific extracellular matrix is isolated from human lung tissue. In some embodiments, the methods further comprise contacting the ventral anterior foregut spheroids with midkine (MDK), semaphorin-3C (SEMA3C), growth/differentiation factor-15 (GDF15), or any combination thereof.

Also disclosed herein are the vascularized proximal lung organoids produced by the methods disclosed herein. In some embodiments, the vascularized proximal lung organoids are embedded in a lung-specific extracellular matrix, optionally wherein the lung-specific extracellular matrix is isolated from human lung tissue.

Also disclosed herein are methods of contacting any of the vLuO or vascularized proximal lung organoids disclosed herein with a perfusion system.

### Methods of Vascularized Intestinal Organoids

Also disclosed herein are methods of producing vascularized small intestine organoids (vHIO). In some embodiments, the method comprises one or more of the methods disclosed in Example 8. In some embodiments, the methods comprise contacting mid/hindgut spheroids disclosed herein with: 1) a BMP pathway inhibitor and VEGF, and optionally with R-spondin and EGF, for a first period of time; and 2) VEGF, and optionally EGF, for a second period of time, thereby differentiating the mid/hindgut spheroids into vHIO. In some embodiments, the vHIO expresses CDX2, GATA4, and CDH17, is negative for SOX2, and comprises a CD31+ vascular bed. In some embodiments, the BMP pathway inhibitor is Noggin, Dorsomorphin, RepSox, LY364947, LDN193189, follistatin, chordin, or any combination thereof, optionally Noggin. In some embodiments, the BMP pathway inhibitor is provided at a concentration that is, is about, is at least, is at least about, is not more than, or is not more than about, 100, 110, 120, 130, 140, 150, 160, 170, 180, 190, or 200 ng/mL, or any concentration within a range defined by any two of the aforementioned concentrations. In some embodiments, the VEGF is provided at a concentration that is, is about, is at least, is at least about, is not more than, or is not more than about, 10, 20, 30, 40, 50, 60, 70, 80, 90, or 100 ng/mL, or any concentration within a range defined by any two of the aforementioned concentrations. In some embodiments, the VEGF is VEGF121 or VEGF165. In some embodiments, the mid/hindgut spheroids are embedded in a basement membrane matrix, step 1) is carried out in a stationary culture, and step 2) is carried out in a culture with shaking. In some embodiments, the first period of time is, is about, is at least, is at least about, is not more than, or is not more than about, 1, 2, 3, 4, or 5 days, optionally 3 days. In some embodiments, the first period of time is at least 3 days. In some embodiments, the second period of time is, is about, is at least, is at least about, is not more than, or is not more than about, 10, 11, 12, 13, 14, 15, 16, 17, 18, 19, or 20 days. In some embodiments, the second period of time is at least 10, 11, 12, 13, 14, 15, 16, 17, 18, 19, or 20 days. In some embodiments, the mid/hindgut spheroids comprise a CD31+ vascular network surrounding the CDX2+/FOXA2+ mid/hindgut epithelium. In some embodiments, the mid/hindgut spheroids are the mid/hindgut spheroids produced by any of the methods disclosed herein.

Also disclosed herein are the vHIO produced by any of the methods disclosed herein.

Also disclosed herein are methods of producing a vascularized colonic organoid (vHCO). In some embodiments, the method comprises one or more of the methods disclosed in Example 8. In some embodiments, the methods comprise contacting mid/hindgut spheroids disclosed herein with: 1) a BMP pathway activator and VEGF, and optionally EGF, for a first period of time, and 2) VEGF, and optionally EGF, for a second period of time, thereby differentiating the mid/hindgut spheroids into vHCO. In some embodiments, the vHCO expresses SATB2 and CDH17, and comprises a CD31+ vascular bed. In some embodiments, the BMP pathway activator is BMP1, BMP2, BMP3, BMP4, BMP5, BMP6, BMP7, BMP8a, BMP8b, BMP10, BMP11, BMP15, IDE1, IDE2, or any combination thereof, optionally BMP2. In some embodiments, the BMP pathway activator is provided at a concentration that is, is about, is at least, is at least about, is not more than, or is not more than about, 50, 60, 70, 80, 90, 100, 110, 120, 130, 140, or 150 ng/mL, or any concentration within a range defined by any two of the aforementioned concentrations, optionally 100 ng/mL. In some embodiments, the VEGF is provided at a concentration that is, is about, is at least, is at least about, is not more than, or is not more than about, 10, 20, 30, 40, 50, 60, 70, 80, 90, or 100 ng/mL, or any concentration within a range defined by any two of the aforementioned concentrations. In some embodiments, the VEGF is VEGF121 or VEGF165. In some embodiments, the mid/hindgut spheroids are embedded in a basement membrane matrix, step 1) is carried out in a stationary culture, and step 2) is carried out in a culture with shaking. In some embodiments, the first period of time is, is about, is at least, is at least about, is not more than, or is not more than about, 1, 2, 3, 4, or 5 days, optionally 3 days. In some embodiments, the first period of time is at least 3 days. In some embodiments, the second period of time is, is about, is at least, is at least about, is not more than, or is not more than about, 10, 11, 12, 13, 14, 15, 16, 17, 18, 19, or 20 days. In some embodiments, the second period of time is at least 10, 11, 12, 13, 14, 15, 16, 17, 18, 19, or 20 days. In some embodiments, the mid/hindgut spheroids comprise a CD31+ vascular network surrounding the CDX2+/FOXA2+ mid/hindgut epithelium. In some embodiments, the mid/hindgut spheroids are the mid/hindgut spheroids produced by any of the methods disclosed herein.

Also disclosed herein are the vHCO produced by any of the methods disclosed herein.

### Methods of Use

In some embodiments, the organoids disclosed herein are transplanted into a mammal, such as a mouse, such as an immunocompromised mouse. In some embodiments, the organoid is transplanted to the kidney capsule of the mammal. In some embodiments, the transplanted organoid grows about 50×, 150×, 200×, 250×, 300×, 400×, 500×, 600×, 700×, 800×, 900×, 1000×, 1100×, 1200×, 1300×, 1400×, or 1500× or at least 50×, 150×, 200×, 250×, 300×, 400×, 500×, 600×, 700×, 800×, 900×, 1000×, 1100×, 1200×, 1300×, 1400×, or 1500× in volume. However, a distinct advantage of some embodiments of the organoids disclosed herein is that they develop vascularization without the need for transplantation into an animal.

Also disclosed herein are methods of screening. In some embodiments, the methods comprise contacting any one of the mesendoderm spheroids, foregut spheroids, mid/hindgut spheroids, ventral anterior gut spheroids, vLuO, vascularized proximal lung organoids, vHIO, and/or vHCO disclosed herein with a compound of interest and assessing a change in phenotype in the cell population or tissue. In some embodiments, the mesendoderm spheroids, foregut spheroids, mid/hindgut spheroids, ventral anterior gut spheroids, vLuO, vascularized proximal lung organoids, vHIO, and/or vHCO disclosed herein are derived from stem cells obtained from a subject. In some embodiments, the subject comprises a disease and the change in phenotype in response to the compound of interest is associated with an improvement of the disease.

### EXAMPLES

### Example 1. Human Fetal Atlas Revealed Organ-Specific EC Gene Signatures During Development

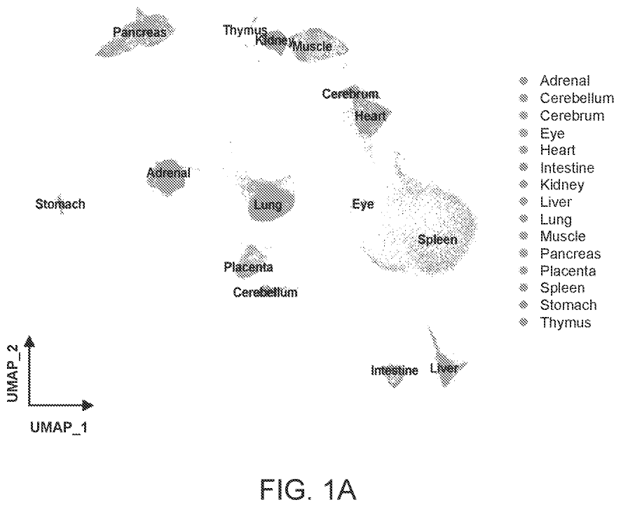

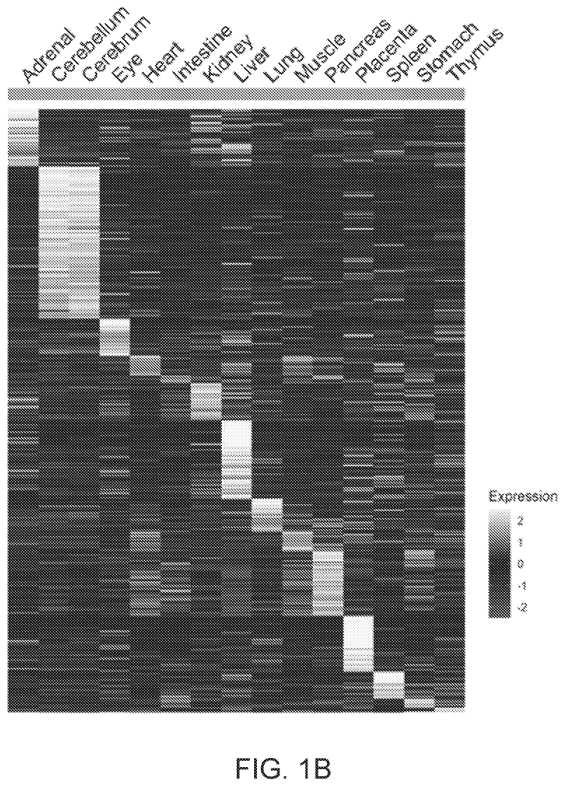

To determine the organ-specific gene expression patterns in ECs, 91 single cell RNA-seq datasets of 15 human fetal organs collected from gestational day 72 to 129 were analyzed. First, ECs from each organ were selected based on high CDH5 expression compared to other cell types, and then significantly enriched genes in different organs were identified (FIG. 1A-B). ECs showed the most tissue specificity in the brain, which contains a highly selective capillary network, the blood brain barrier. Human lung and intestine also showed a set of organ-specific genes such as FENDRR and HPGD for the lung, and NKX2-3 and LTBP1 for the intestine.

### Example 2. Co-Differentiation of the Endoderm and Mesoderm from Human iPSCs

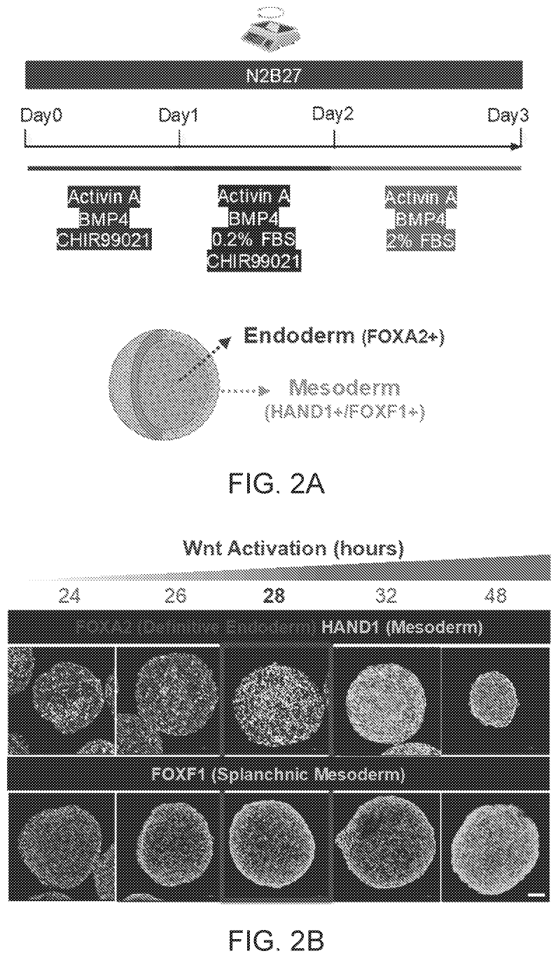

To recapitulate the gut tube organogenesis in vitro, a 3D suspension culture system to differentiate endoderm and mesoderm concomitantly from human iPSCs was established. By fine-tuning the dosage and timing of Wnt, Nodal (or TGF-b), and BMP activation, mesendoderm spheroids containing both definitive endodermal (DE, FOXA2+) and lateral plate mesoderm (LPM, HAND1+) populations were generated (FIG. 2A). It was also determined that ˜28 hours of the Wnt activation was a good duration for generating appropriate amounts of both DE and LPM populations from the iPSCs (approximately 1:1) (FIG. 2B). LPM derived splanchnic mesoderm (SM, FOXF1+) further gives rise to the mesenchyme surrounding the primitive gut tube. The SM will then differentiate into the connective tissue, smooth muscle cells (SMCs), and blood vessels of the endodermal organs.

### Example 3. Co-Development of the Vasculature. SM, and the Gut Tube

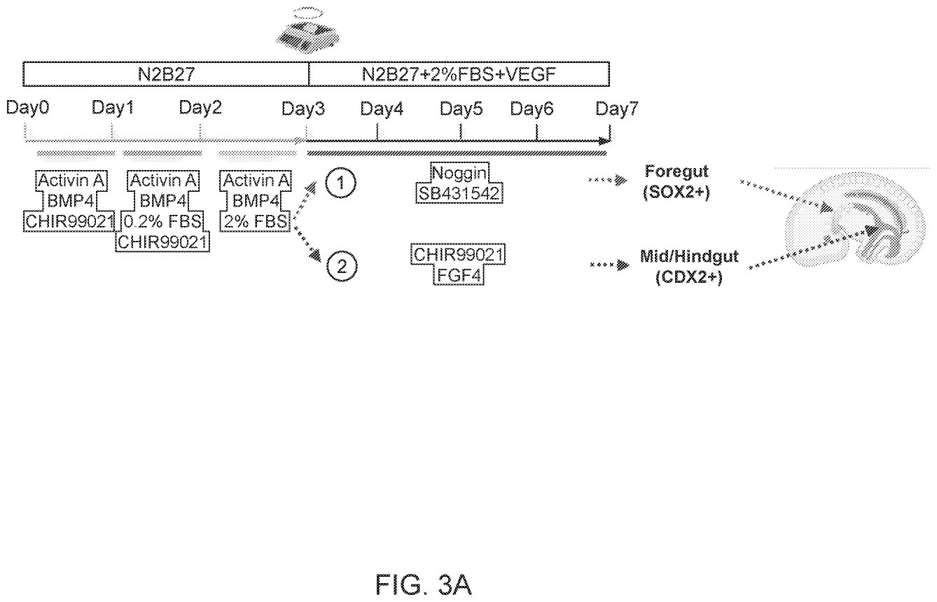

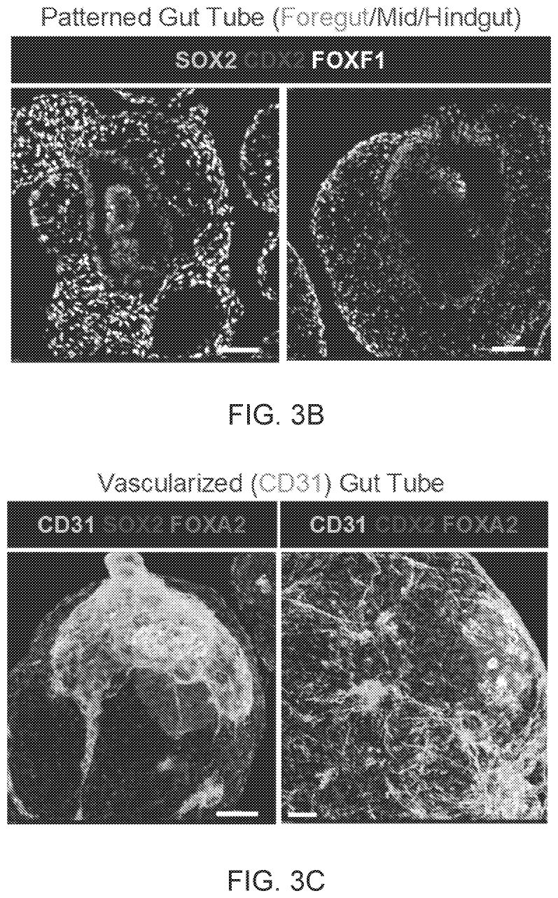

To form various organs properly, the endoderm transforms into a primitive gut tube and is then regionalized along the dorsal-ventral (D-V) and anterior-posterior (A-P) axis into foregut, midgut, and hindgut under the control of various signaling pathways: BMP and TGF-b inhibition facilitate foregut formation, while Wnt and FGF activation facilitate mid/hindgut development. Here, these signals were applied after the generation of the mesendoderm spheroids on day 3 (FIG. 3A), and either foregut (SOX2+) or mid/hindgut (CDX2+), which were both tightly surrounded by SM (FOXF1+), were successfully patterned (FIG. 3B). Additionally, by introducing vascular endothelial growth factor (VEGF, 50 ng/mL), an angiogenic factor, into the differentiation system from day 3 to day 7, a significant amount of a vascular network (CD31+) surrounding the FOXA2+ epithelial layer was successfully generated (FIG. 3C).

### Example 4. Vascularization of Distal Lung Organoids

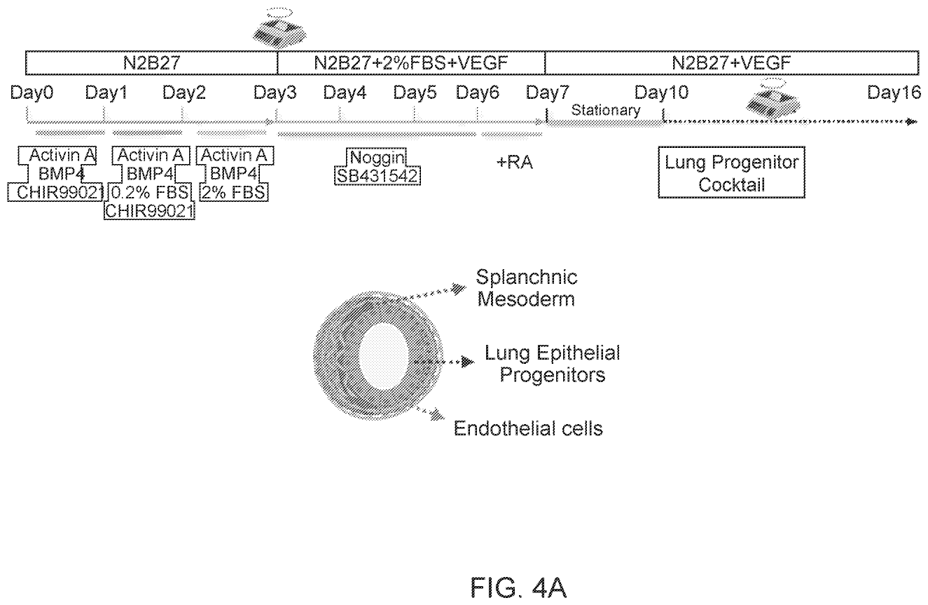

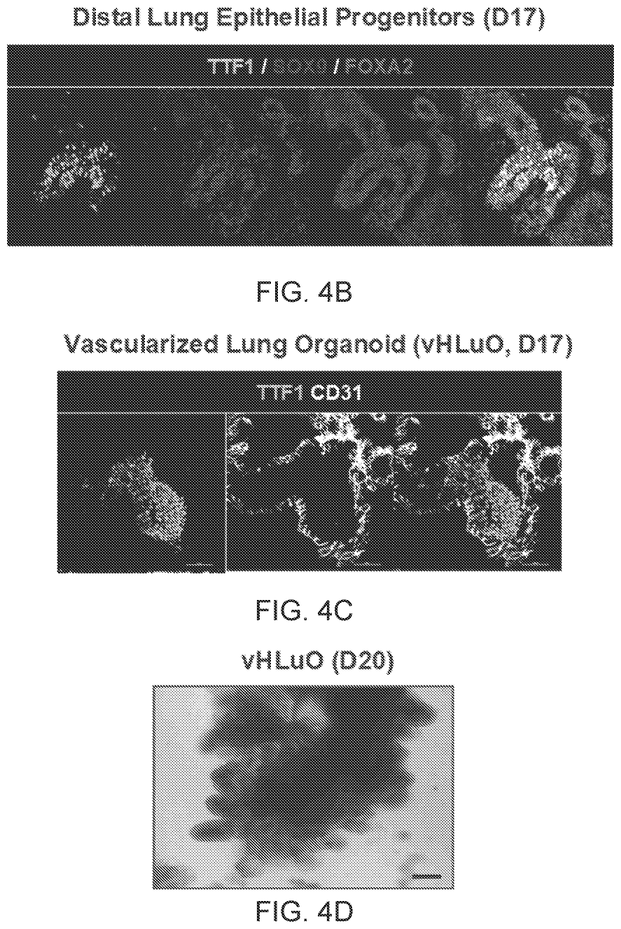

Ventral anterior foregut (vAFG) gives rise to respiratory lineages (TTF1+, also known as NKX2.1+) and further specified into distal lung tip (SOX9+) or proximal airway (SOX2+). Given the functional significance of the alveolar-capillary interface in gas exchange, a protocol to vascularize distal lung organoids was developed. On differentiation day 7, foregut spheroids were embedded into a collagen-Matrigel mixture for further specification. After three days of stationary culture in an ultralow attachment plate, the embedded spheroids were placed on an orbital shaker and incubated with lung progenitor cocktail (for example, but not necessarily: CHIR99021, FGF7, FGF10, all-trans retinoic acid (ATRA), BMP4) and VEGF to promote both lung epithelial and endothelial growth as a vascularized lung organoid (vLuO) (FIG. 4A). After seven days, the TTF1 (NKX2-1)-expressing lung epithelial progenitors were observed and stained positive for distal lung marker SOX9 (FIG. 4B). Intriguingly, the lung epithelial layer was surrounded by the vasculature (FIG. 4C), which resembles the patterning of the alveolar-capillary interaction in human distal lung. Notably, the distal vLuO also showed branching morphogenesis, possibility due to the high FGF10 secreted by the surrounding mesenchyme (FIG. 4D). This phenotype came on much earlier compared with previous lung organoid generation protocols, which require ˜1.5 or 2 months of culture.

### Example 5. Improving the Patterning, Lung-Specificity, and Perfusion of the iPSC-Derived vLuO

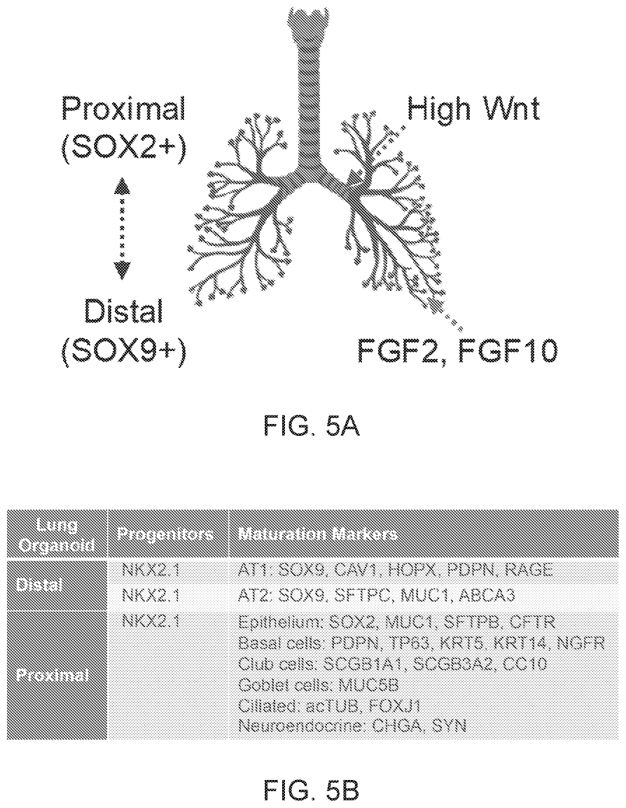

Proximal-distal (airway-alveolus) patterning of the vLuO: As described herein, vLuO with distal lung progenitor (SOX9+) were generated. The TTF1+ lung progenitor lineage will be further purified, and proximal lung (airway) organoids will be vascularized through proximal-distal patterning of the respiratory system. The necessity or adverse effect of adding exogenous BMP, FGF, Wnt, and/or retinoic acid modulators during early lung specification stages will be assessed to further improve the purity of the lung epithelial progenitors while maintaining well-organized vascular beds with lung-specific gene signatures and relevant functionalities. Wnt activation (e.g. Wnt2 and Wnt2b secreted by lung mesenchyme) prompts rapid distal lung patterning, whereas FGF2 and FGF10 specify proximal lung lineages (FIG. 5A). These developmental cues will be followed to further specify the systems provided herein to resemble proximal (airway) vLuO. It is also recognized that, contrary to previously reported protocols for generating proximal vs. distal lung epithelium, the co-development system disclosed herein may already have intrinsic growth factors produced by mesoderm derived mesenchyme and ECs, such as VEGF, FGF, and TGF-b. Thus, the organoids are stained to determine the type of growth factors being expressed intrinsically, and then the protocol is adjusted by reducing or eliminating small molecules in the 3D co-differentiation system. Additionally, given the close association of the capillary endothelium and alveolar type 1 (AT1) cells within thin regions of the gas-exchange surface, and the critical role of the Yap/Taz signaling in driving and maintaining AT1 identity, it is tested if the endothelial lineage and/or Yap/Taz activation in the distal vLuO generates AT1 cells with a flattened morphology. AT1 cells cover 95% of the alveolar system but are still missing from other lung organoid systems.

Generation of vLuO using lung-specific extracellular matrix (ECM): ECM composition is tissue-specific, and ECM derived from different tissues can provide physical and biochemical cues that drive important processes for organ formation and maintaining tissue homeostasis. vLuO are embedded in ECM extracted from decellularized human lung tissue, and the organoids are examined for morphological changes, expression of maturation marker genes (FIG. 5B), and lung-specific genes in both ECs and epithelial cells, in comparison to the commonly used collagen and Matrigel extracellular matrices. The stiffness of the ECM extracted from adult lung tissue will also be modulated to resemble the fetal lung ECM early in development through biochemical approaches. Additionally, using scRNA-seq datasets from multiple human fetal organs, the composition and key components of the lung/gut tube-specific ECMs at different developmental stages are determined.

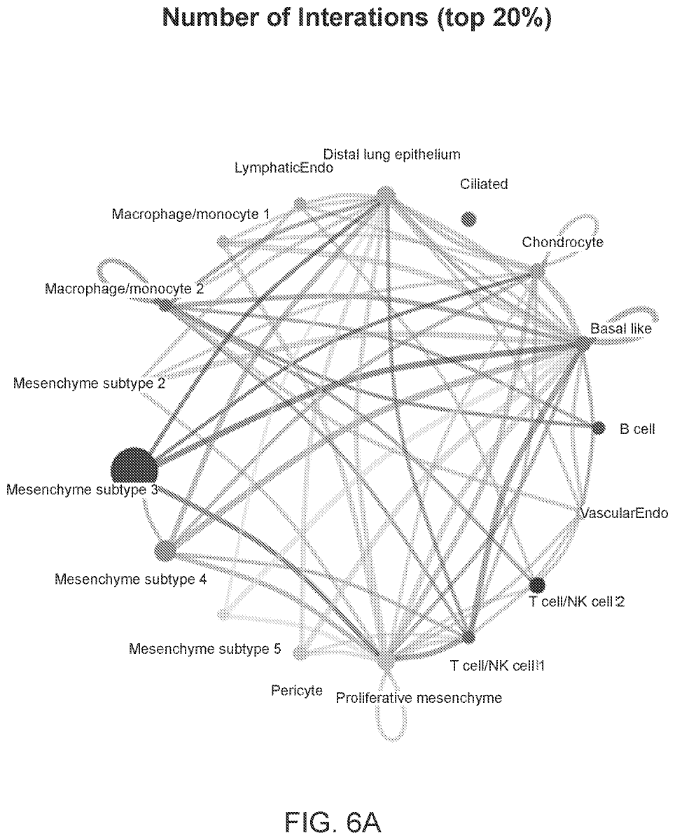

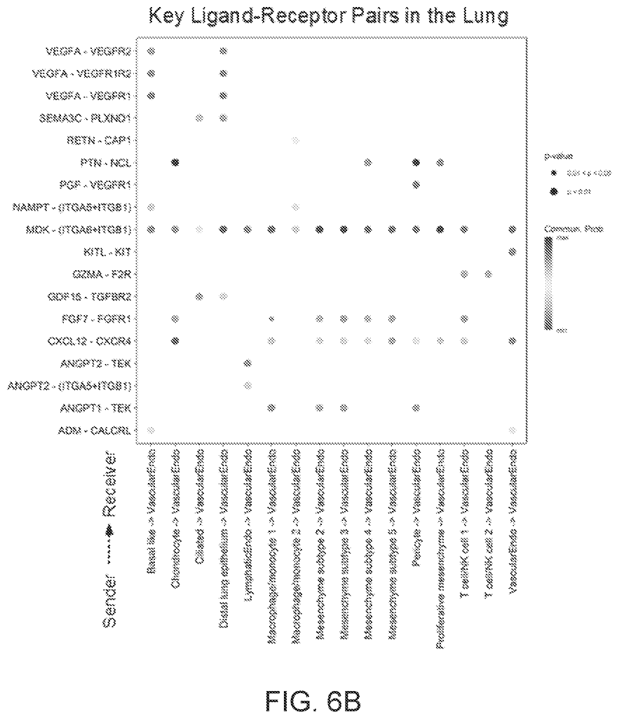

Introducing additional growth factors to further specify lung EC lineage: To determine the key growth factors that specifically signal through human lung ECs during development, CellChat (available on the World Wide Web at cellchat.org) was utilized to analyze ligand-receptor interaction between ECs and surrounding cell types based on published scRNA-seq datasets. Extensive cell-cell communication during the development of human fetal lung was found (FIG. 6A-B). The VEGFA-VEGFR interaction was consistent with previous findings, indicating the robustness of the approaches provided herein. Notably, vascular ECs receive midkine (MDK, an angiogenesis factor) from various cell types in the lung, as well as SEMA3C (Semaphorin-3C) and GDF15 (growth/differentiation factor-15) from distal lung epithelium. The role of these exogenous growth factors in the co-differentiation systems provided herein is tested and the lung specificity of the ECs (e.g. expression of FENDRR and HPGD) as well as maturation and patterning of the vLuO is assessed.

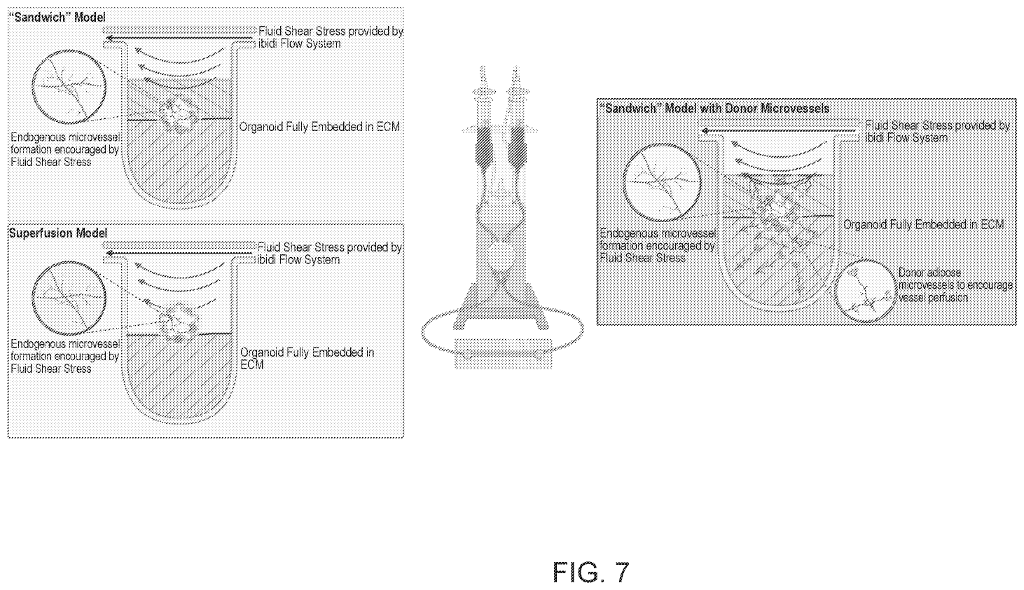

Improving the growth, patterning, and long-term maintenance of vLuO via an in vitro vascular perfusion system: to date, perfusable vasculature within organoids has only been demonstrated through transplantation into host animals, where native vasculature penetrates the ectopic implant. However, the reliance on an animal host limits both the scalability and translation of organoid-based approaches, particularly for in vitro applications. Thus, we introduce circulation and perfusion into the vLuO system. Devices such as the shear stress perfusion system from ibidi, which has been used to apply flow in 3D organoids, combines bona fide native microvessels isolated from human adipose tissue to establish a perfused, dynamic, microvasculature in a 3D tissue space (FIG. 7). Upon perfusion, maturation markers (FIG. 5B), epithelium-endothelium patterning, alveolar barrier function, and the long-term maintenance of the vLuO are assessed. Additionally, these in vitro perfused vLuO are compared to the ones that are transplanted in vivo under the kidney capsule in mice.

### Example 6. Using vLuO as a Novel Platform to Study FOXF1 Mutations

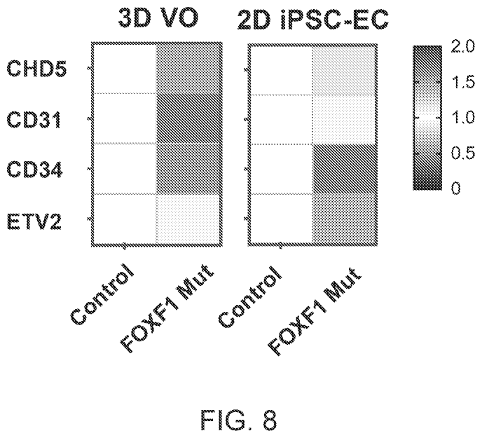

Patients with a FOXF1 mutation usually presents multiple congenital malformations accompanied by respiratory distress and pulmonary hypertension. Common clinical phenotypes include alveolar capillary dysplasia (ACD), misalignment of pulmonary vein (MPV), and pulmonary lymphangiectasia. Most patients also present non-pulmonary abnormalities in organs of the cardiovascular, gastrointestinal, and urogenital systems. Previous study using mouse models showed that during early fetal development (E8.5-E9.5), Foxf1 expression is confined to the splanchnic mesoderm surrounding the entire primitive gut tube. At E11.5, Foxf1 expression was observed in pulmonary ECs and mesenchyme. At E12.5, Foxf1 expression was also seen in visceral smooth muscle cells (SMCs) and mesenchyme surrounding the esophagus, trachea, bronchi, stomach, and intestine. Given the significant role of FOXF1 in mesoderm development, it is interesting that FOXF1 mutation only affects blood vessels in certain organs such as lung, heart, and intestine, but not other organs that arise from the gut tube. Thus, to further understand the role of FOXF1 mutant ECs, SMCs, and mesenchyme in causing vascular abnormalities in an organ-specific manner, FOXF1-mutant iPSC lines will be used. For example, FOXF1-mutant iPSC lines can be isolated from patients with ACD and MPV. Given the high expression level of FOXF1 in ECs, age/gender matched control iPSCs and FOXF1-mutant iPSCs were differentiated into ECs in 2D, as well as generic blood vessel organoids (VO) in 3D. Methods of making VO can be found in PCT Publication WO 2018/229251, which is hereby expressly incorporated by reference in its entirety. It was found that most of the EC marker genes were downregulated in FOXF1 mutant lines compared with control (FIG. 8). Functionally, cell division, proliferation, and ECM production will be examined based on previously reported genes downstream of FOXF1. The methods disclosed herein are used to co-differentiate ECs in a lung-specific microenvironment, and how EC deficiency affects surrounding cells during lung development is determined. Aforementioned genes and functions are tested in vLuO, and by comparing with generic VO, the genes and pathways that are more profoundly impaired in the vLuO, when multiple cell types (SMC, mesenchyme, etc.) are being affected by the FOXF1 mutation, is assessed. Because FOXF1 acts through STAT3 to induce neonatal lung angiogenesis and Foxf1 mutant mice (S52F) showed disrupted STAT3-FOXF1 interaction leading to the inhibition of Stat3 transcription, the activation of the STAT3 pathway in ECs is examined, and whether any of the downstream genes are specifically dysregulated in ECs from the vLuO, but not in generic VO, is assessed. Additionally FOXF1 ChIP-Seq data showed that FOXF1 directly binds to promoter and introns of Wnt2, Wnt11, and Wnt5a. Given the significant role of the Wnt pathway in lung epithelial growth and specification, the potentially impaired interaction of the FOXF1 mutant ECs and epithelium in vLuO is also studied.

### Example 7. Translation of the Co-Development System to Vascularize Other Endodermal Organoids

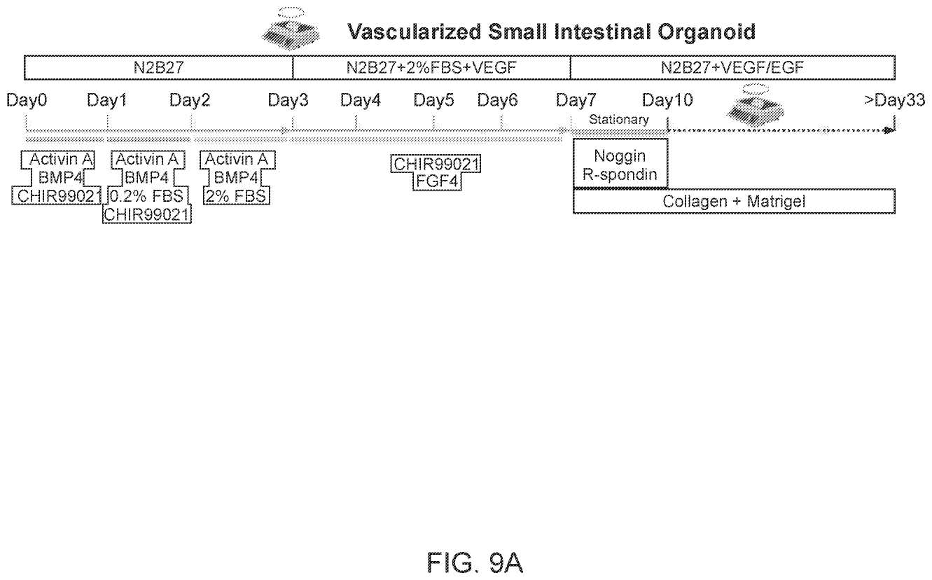

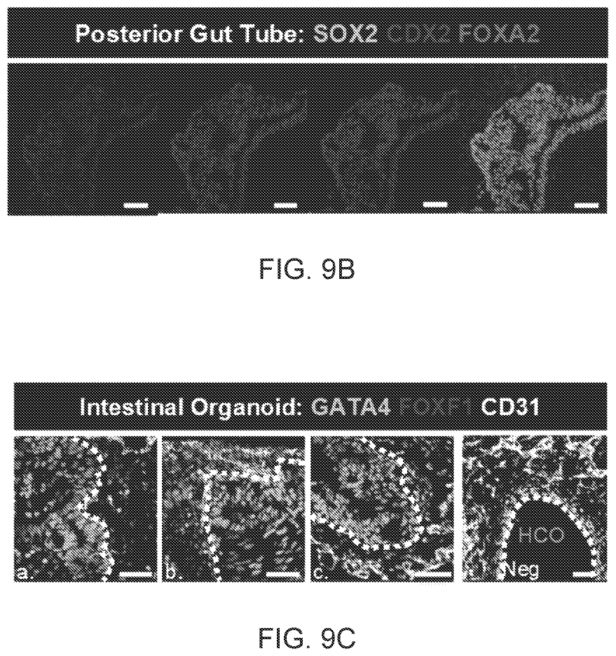

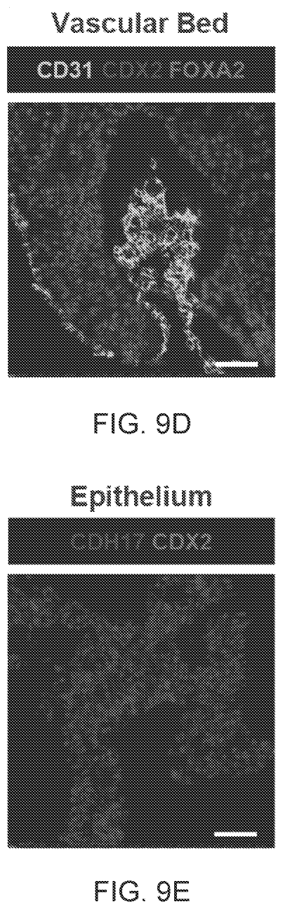

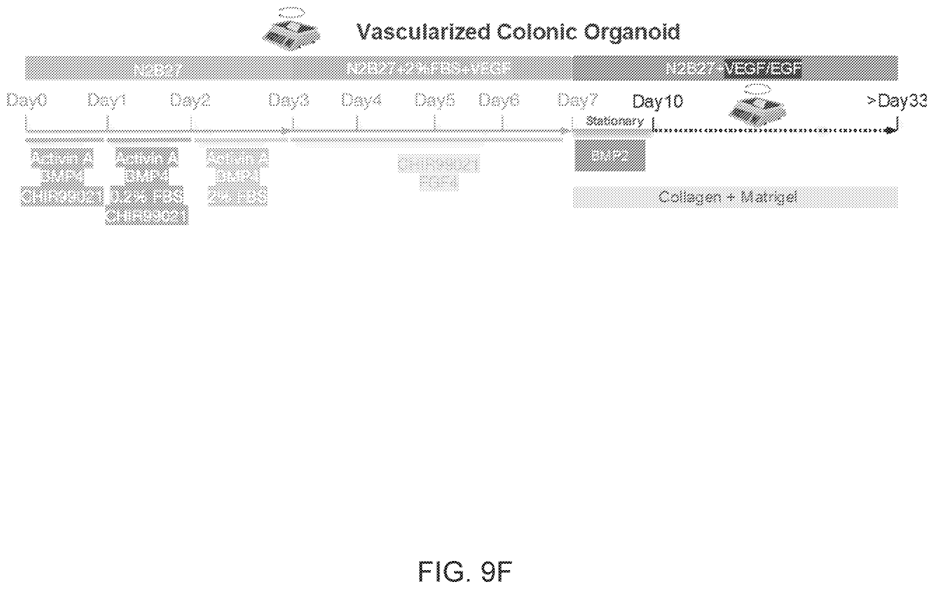

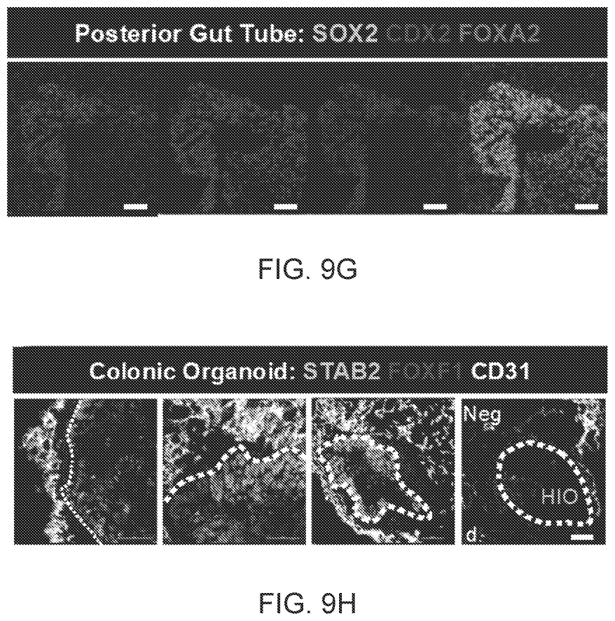

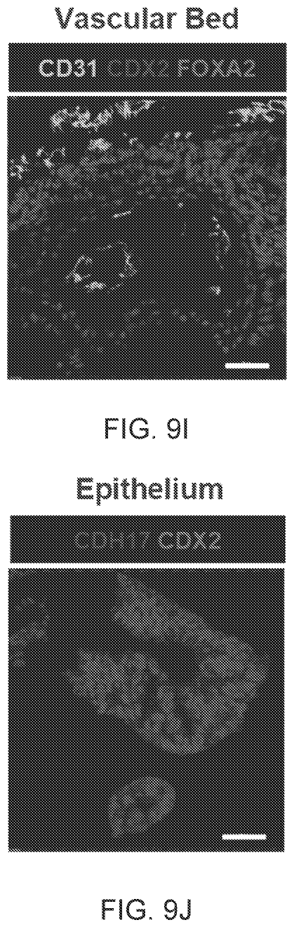

In addition to generating vLuO, the co-development systems disclosed herein can be translated to populate midgut and hindgut derived organs, such as the small intestine and colon, with vasculature. Based on previous protocols for generating small intestinal organoids (HIOs), mid/hindgut spheroids were embedded into collagen and Matrigel and cultured with a BMP pathway inhibitor for three days to differentiate towards the proximal regions of the mid/hindgut (FIG. 9A). After twenty days, the organoids kept their mid/hindgut identity (FIG. 9B, SOX2-CDX2+) and expressed proximal small intestinal marker GATA4 surrounded by SM (FIG. 9C) and vascular bed (FIG. 9D), as well as general intestinal marker CDH17 (FIG. 9E). Notably, previously published protocols showed sustained GATA4 expression only after in vivo transplantation, indicating that the co-differentiation system herein may provide relevant cell types and microenvironment to accelerate the maturation process in vitro.

In contrast to HIO, mid/hindgut spheroids were also cultured with a BMP pathway activator for three days to vascularize human colonic organoids (HCOs) (FIG. 9F). Twenty days of differentiation introduced a vascular bed (FIG. 9G) into the SATB2/CDH17 positive colonic organoids (FIG. 9G-H, J). The system is further matured by fine-tuning the time points for BMP inhibition for small intestinal organoids, testing intestine-specific ECM such as alginate, and incorporating relevant cell types such as immune cells and lymphatic cells. The maturation markers of the intestinal cell types are characterized, and the expression of intestinal specific makers in the ECs, such as NKX2-3 and LTBP1, is determined. The resulting vascularized HIO (vHIO) and vascularized HCO (vHCO) are utilized to study EC fate determination in these organ systems, and are also used as a negative control when characterizing lung-specific features of the ECs in vLuO. Interestingly, intestinal malrotations were also seen in some patients with FOXF1 mutations, and therefore, vHIO and vHCO are also used to study the role of FOXF1 in causing intestinal abnormalities.

### Example 8. Generation of Vascularized Gut-Tube Derived Organoids in a 3D Suspension Culture System

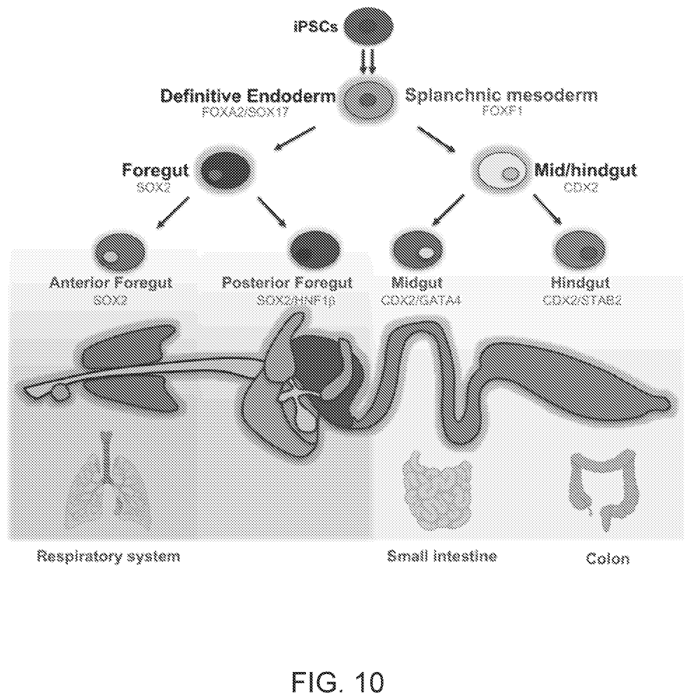

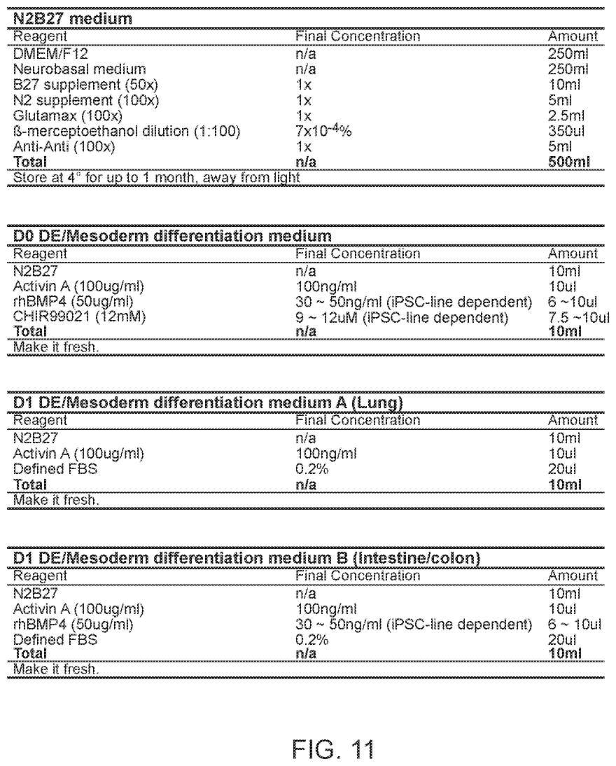

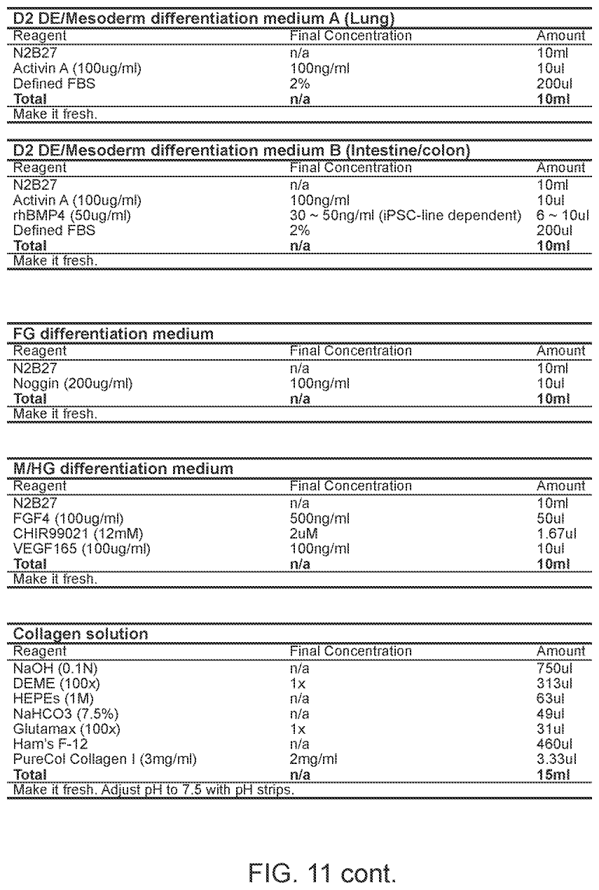

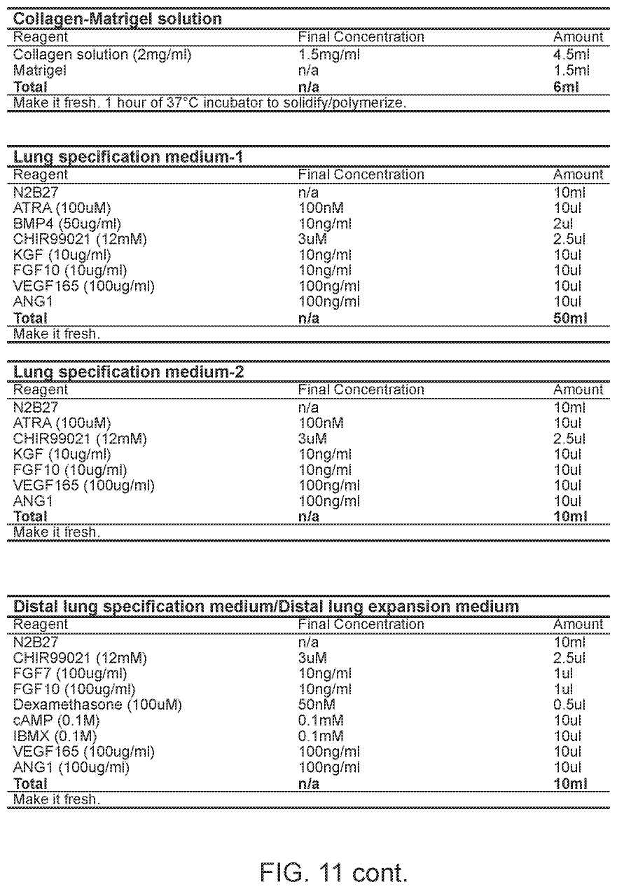

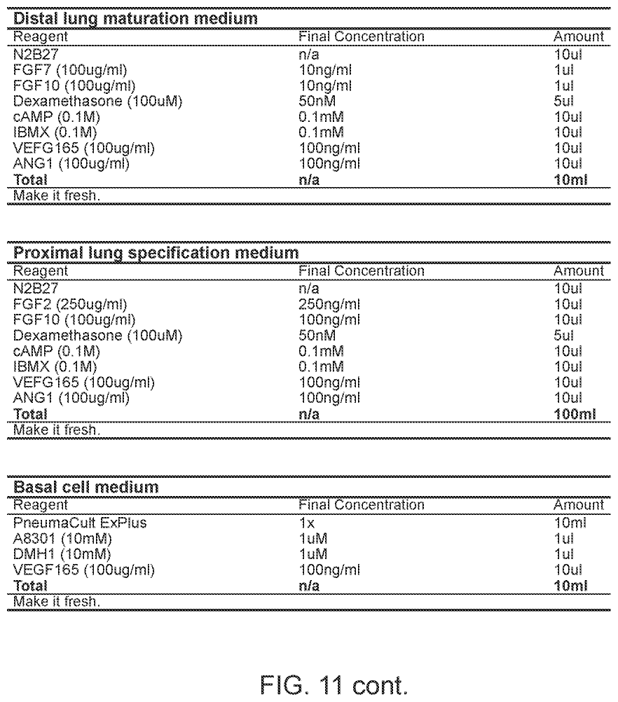

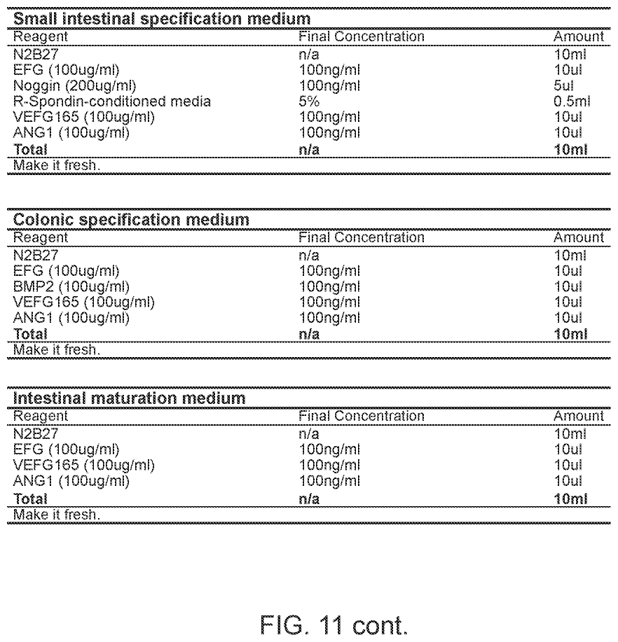

Exemplary media compositions used herein are depicted in FIG. 11.

### 1. Concomitant Generation of Definitive Endoderm and Mesoderm (Day 1-2)

a) At Day −1, dissociate human iPSCs or human ESCs into single cells.
b) Using an Aggrewell plate (StemCell), or other compatible cell collection plate, generate spheroids of the pluripotent cells. Note: the number of cells in each microwell should be between 500-2000 cells. Accordingly, the spheroid diameter range should be from 50-200 μm.
c) Culture the cells in steady condition (37° C. cell incubator) overnight. Note: usually, the spheroid will form at day 0. If the spheroid morphology is still loose, culture for another one day.
d) At Day 0, resuspend the formed spheroids and transfer them into a 5 mL tube.
e) Let the spheroids settle for 5 minutes at room temperature and wash with 2 mL plain N2B27 medium.
f) Resuspend the spheroids with DO DE/Mesoderm differentiation medium and evenly distribute them into ultralow attachment 6-well plates, with 3 mL medium in each well. Put the plate on an orbital shaker at the speed of 90-110 rpm in a cell culture incubator and culture for 24-32 hours. Note:
   1) if using an Aggrewell 400 plate, one microwell of spheroid (around 400 spheroids) can be distributed into 3 wells of an ultralow attachment 6-well plate so that spheroids won't fuse at rotating speeds above 90 rpm.
   2) Depends on each hESC/iPSC line, if the mesoderm induction rate is low (HAND1 positive percentage), the culture time can be increased from 24 hours to 28-32 hours.
g) At Day 1, transfer the Day 0 spheroids into 5 mL tubes and aspirate the supernatant. Add D1 DE/Mesoderm differentiation medium A to generate Lung DE/Mesoderm spheroids or D1 DE/Mesoderm differentiation medium B to generate intestinal/colonic spheroids. Culture on the shaker for 24 hours.
h) At Day 2, transfer the Day 1 spheroids into 5 mL tubes and aspirate the supernatant. Add D2 DE/Mesoderm differentiation medium A to lung DE/Mesoderm spheroids. Add D2 DE/Mesoderm differentiation medium B to intestinal/colonic spheroids. Incubate on the shaker for 24 hours.
   2. Generation of Foregut (FG) or Mid/Hindgut (M/HG) with Endothelial Cell (EC) Progenitors (Day 3-6)

(1) Foregut with endothelial cell progenitors:
a) At Day 3, transfer the Day 2 lung DE/Mesoderm spheroids into 5 mL tubes and aspirate the supernatant. Add FG differentiation medium and incubate on the shaker for 24 hours.
b) At Day 4, transfer the Day 3 spheroids into 5 mL tubes and aspirate the supernatant. Add FG differentiation medium and incubate on the shaker for 24 hours.
c) At Day 5, transfer the Day 4 spheroids into 5 mL tubes and aspirate the supernatant. Add FG differentiation medium and incubate on the shaker for 24 hours.
d) At Day 6, transfer the Day 5 spheroids into 5 mL tubes and aspirate the supernatant. Add FG differentiation medium and incubate on the shaker for 24 hours. Afterwards, proceed to Step 3 for lung lineages.

(2) Mid/hindgut with endothelial cell progenitors:
a) At Day 3, transfer the Day 2 intestinal/colonic spheroids into 5 mL tubes and aspirate the supernatant. Add M/HG differentiation medium and incubate on the shaker for 24 hours.
b) At Day 4, transfer the Day 3 spheroids into 5 mL tubes and aspirate the supernatant. Add M/HG differentiation medium and incubate on the shaker for 24 hours.
c) At Day 5, transfer the Day 4 spheroids into 5 mL tubes and aspirate the supernatant. Add M/HG differentiation medium and incubate on the shaker for 24 hours.
d) At Day 6, transfer the Day 5 spheroids into 5 mL tubes and aspirate the supernatant. Add M/HG differentiation medium and incubate on the shaker for 24 hours. Afterwards, proceed to Step 5 for intestinal lineages.
   3. Generation of EC Progenitors Underlying Lung Progenitors from Foregut (Day 7-21)

### (1). Droplet-Based Methods

a) At Day 7, prepare dimpled Parafilm substrate for generation of Matrigel/collagen droplets by layering a square of Parafilm over an empty tip tray for size 20 μL tips.
b) Make a grid of 4×4 or 3×8 dimples and trim the Parafilm with sterile scissors to a small square containing this grid. Place the square of Parafilm into a 60 mm tissue culture dish.
c) Using a cut 200 μL tip, transfer FG spheroids from Step 2(1) one by one to each dimple in the Parafilm.
d) Remove excess media from each tissue by carefully sucking off the fluid with an uncut 200 μL tip.
e) Immediately add droplets of Matrigel/collagen to each aggregate by dripping approximately 30 μL onto each tissue so that the droplet fills the Parafilm dimple.
f) Position each aggregate in the center of the droplet using a syringe needle to move the tissue within the droplet.
g) Place the 60 mm dish containing droplets on Parafilm back into a 37° C. incubator and incubate for 1 hour to allow the Matrigel/collagen to polymerize.
h) Remove Matrigel droplets from parafilm by first using sterile forceps to turn the Parafilm sheet over and agitating until the droplets fall off the sheet into a 6-well ultralow attachment plate. Any remaining droplets can be removed by flushing with medium. Keep 11-14 droplets per well for 6-well plate.
i) Continue culturing organoid droplets in incubator stationarily for 3 days (Day 7-9). Add 3 mL of lung specification medium-1 and change the medium every other day.
j) After 3 days, put the plate onto the orbital shaker at a speed of 90-110 rpm for further culture (Day 10-21). Change to the lung specification medium-2 and refresh the medium every other day. Afterwards, proceed to Step 4 for either distal or proximal lung differentiation.

### (2). Transwell-Based Method

a) Collect 7-10 foregut organoids and quickly spin-down in a 1.5 ml tube.
b) Resuspend the foregut organoids with 20-50 ul Matrigel/collagen solution and add them onto the insert of 24-well transwell plate.
c) Plate the plate back into the 37° C. incubator and incubate for 1 hour to allow the Matrigel/collagen to polymerize.
d) Add lung specification medium-1 to the transwell plate from Day 7˜Day 9. 600 ul to the well and 150 ul to the insert.
e) Change to lung specification medium-2 and refresh every other day (Day 10-Day 21). Afterwards, proceed to Step 4 for either distal or proximal lung differentiation.
   Note:
      1) Both droplet- and transwell-based method yield similar lung progenitor organoids in terms of marker expressions. Transwell-based method gives lung organoid more space to grow, which exerts different morphology.

### 4. Generation of Vascularized Distal or Proximal Lung Organoids (Day 21-60)

### (1) Vascularized Distal Lung Organoid (Day 21-60)

a) At Day 21, switch from the lung specification medium to distal lung specification medium for lung progenitor organoids generated from Step 3. Change the distal lung specification medium every other day.
b) At Day 32, change the medium to distal lung maturation medium. Change the medium every other day.
c) At Day 40, change the medium to distal lung expansion medium and change the medium every other day until Day 45-60.

### (2) Vascularized Proximal Lung Organoid (Day 17-40)

a) At Day 21, switch from the lung specification medium to proximal lung specification medium for lung progenitor organoids generated from Step 3. Change the proximal lung specification medium every other day.
b) At Day 35, either keep culturing or switch the medium to basal cell medium for another 10 days.

### 5. Generation of Vascularized Small Intestinal Organoid (Day 7-30)

a) At Day 7, generate Matrigel/Collagen droplet or transwell-based method containing mid/hindgut spheroid from Step 2 (2). Use the same method described in Step 3A (a-j) or Step 3B (a-d).
b) Add small intestinal specification medium and change the medium every other day from Day 7˜Day 9.
c) From Day 10, change to the intestinal maturation medium every other day until Day 30.

### 6. Generation of Vascularized Large Intestinal (Colonic) Organoid (Day 7-30)

a) At Day 7, generate Matrigel/collagen droplet or transwell-based method containing mid/hindgut spheroids from Step 2(2). Use the same method described in Step 3 A (a-j) or Step 3B (a-d).
b) Add colonic specification medium and change the colonic specification medium every other day from Day 7-Day 9.
c) From Day 10, change to the intestinal maturation medium every other day until Day 30.

### Example 9: Generation of Vascularized Distal Lung Organoid. Vascularized Human Intestinal Organoid, and Vascularized Human Colonic Organoid

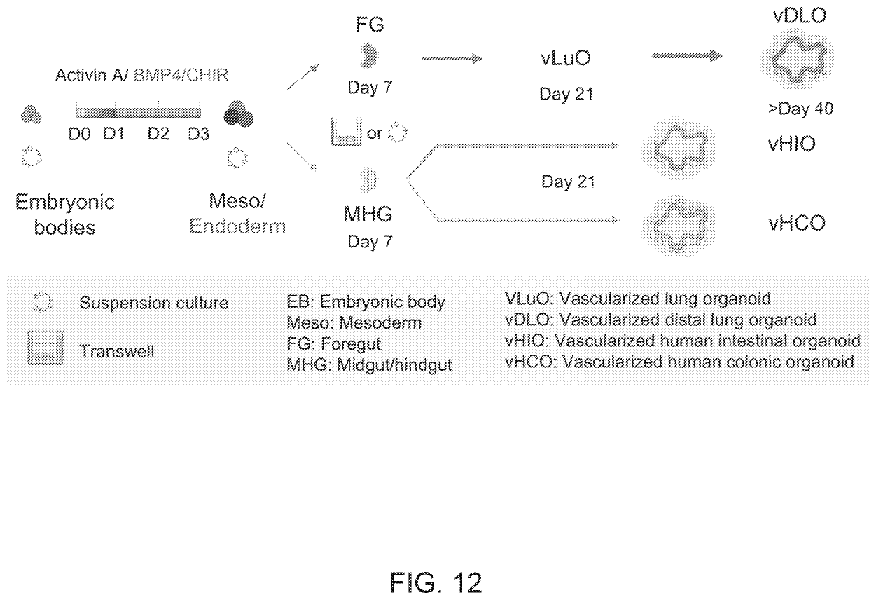

Human pluripotency stem cells were firstly aggregated into embryonic bodies using Aggrewell and exposed to Nodal, BMP, and Wnt stimulators in a continuous or pulsatile manner within the first three days (FIG. 12). The induction of mesoderm and endoderm were characterized by FACS, Immunostaining, and single-cell RNA-seq. Spheroids containing both mesoderm and endoderm were regionalized into foregut or midgut/hindgut respectively until Day 7. Proper splanchnic mesoderm and vascular progenitors emerged at this stage. Different gut tube miniatures were embedded in ECM-transwell insert and differentiated into vascularized lung, intestinal, and colonic organoid.

### Example 10: Generation of Vascularized Gut Tube Using a Co-Developmental Strategy

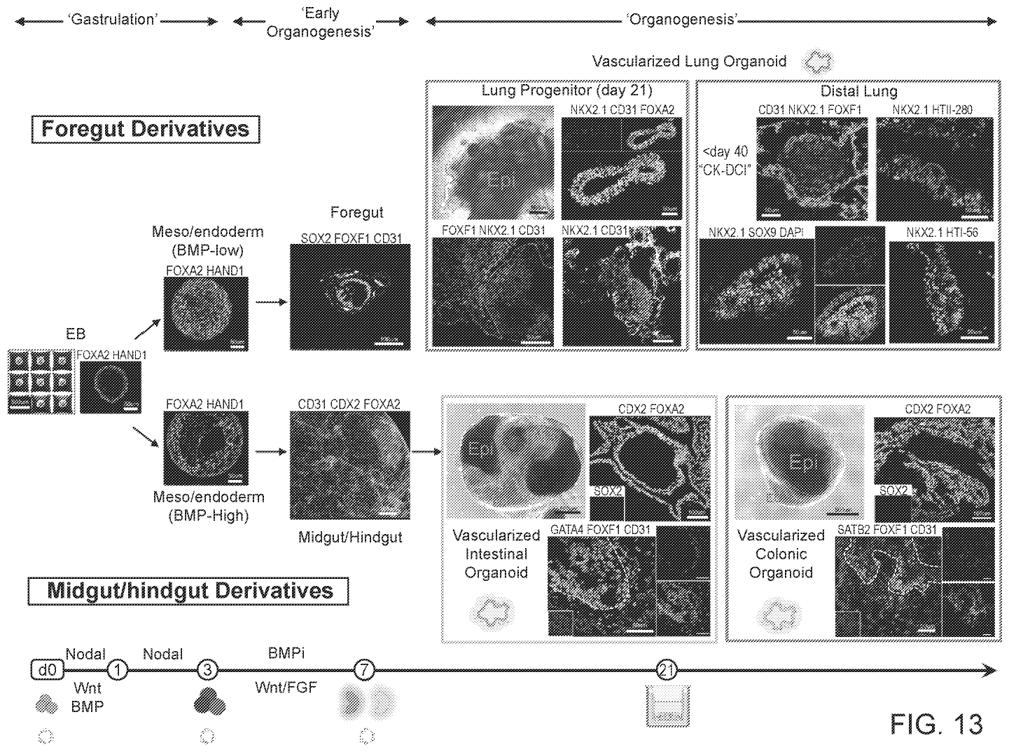

The mesoderm (HAND1+) and endoderm (FOXA2+) were co-emerged through modulation of Wnt and BMP signaling during gastrulation stage (FIG. 13). Meso/endoderm spheroids were specified towards foregut (SOX2+) or mid/hindgut (CDX2+) with the development of endogenous endothelial progenitors (CD31+). Foreguts miniatures preserved the potentials to generate lung progenitors and distal lung organoid under the lung differentiation cocktails. The lung bud epithelium were wrapped by FOXF1+ splanchnic mesoderm and vasculature. Under appropriate differentiation conditions, midgut/hindgut organoids were able to form vascularized intestinal (CDX2+/GATA4+) or colonic (CDX2+/SATB2+) organoids.

### Example 11: Wnt and BMP Signals are Determinant for Mesoderm Vs. Endoderm Induction

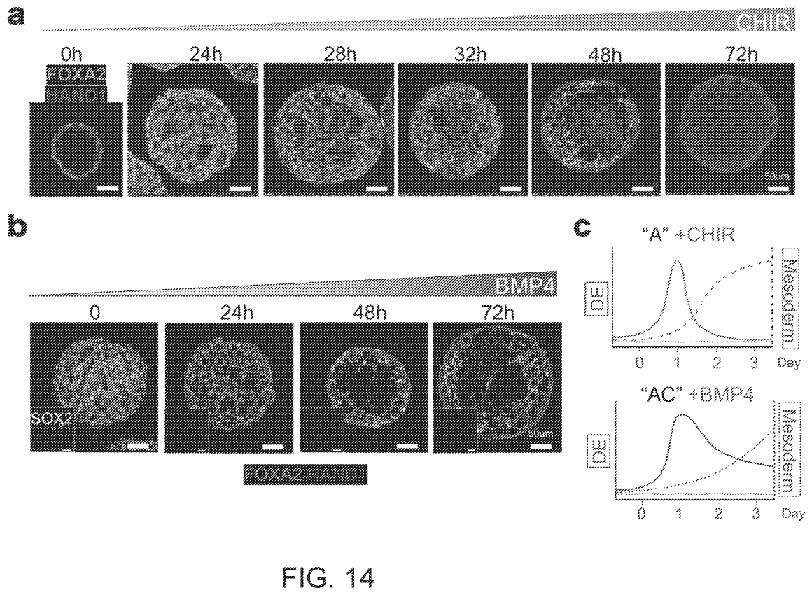

Activation of Wnt by CHIR99201 under different durations drastically changed mesoderm/endoderm ratio during the first three days (FIG. 14A). Prolonged treatment with BMP4 increased mesoderm/endoderm ratio (FIG. 14B). FIG. 14C depicts the change in the mesoderm/endoderm ratio in the presence of activin A, CHIR99201 (top figure) or activin A, CHIR99201 and BMP4 (lower figure) over the first three days. FOXA2 staining in green is a marker of Definitive Endoderm (DE); HAND1 staining in red is a marker of Lateral plate mesoderm; A: Activin A; C: CHIR99201.

### Example 12: Acquirement of Organ-Specific EC Gene Signatures in Vascularized Lung Organoids

Identification of human lung EC specific markers from human cell atlas maps (FIG. 15A). Verification of human lung EC marker HPGD in several human fetal tissues (FIG. 15B). HPGD was exclusively expressed in ECs from vascularized lung organoids over intestinal organoids (FIG. 15C). Staining: RNA-scope.

### Example 13: Characterization of Day 3 Meso/Endoderm Organoids by Single-Cell RNA-Seq

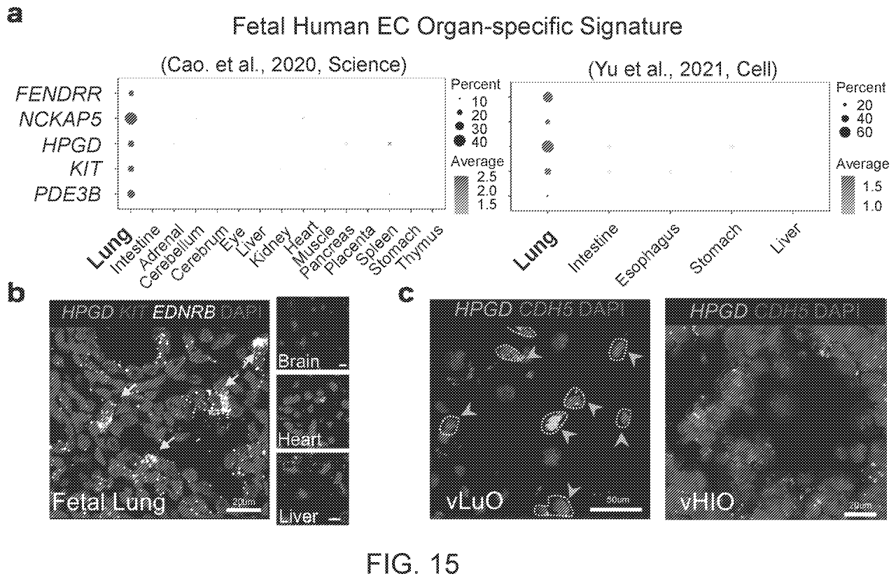

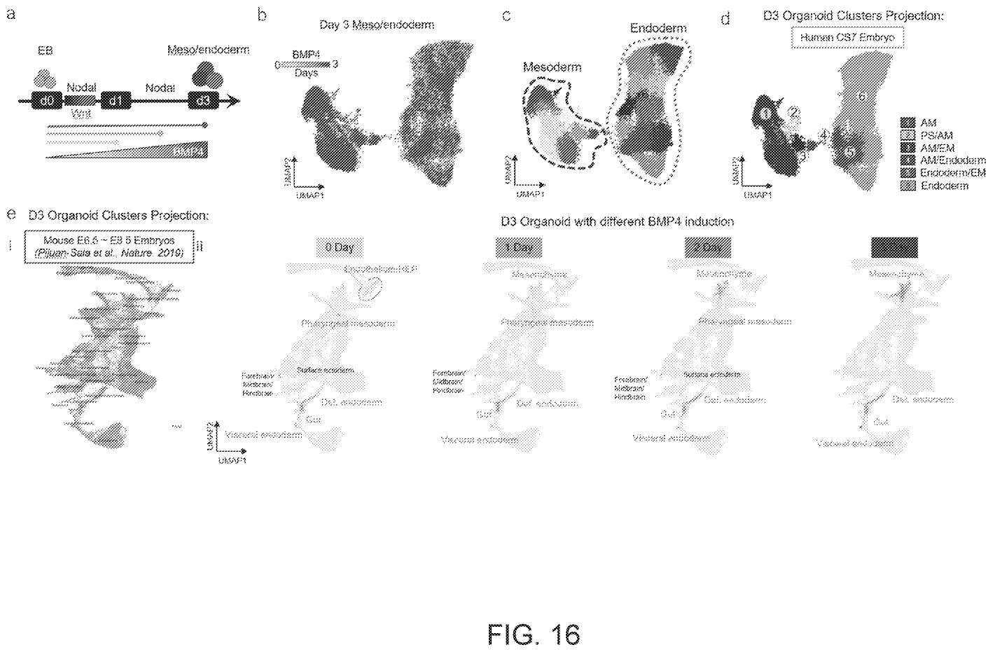

Embryoid bodies (EBs) receiving various duration of BMP4 stimulation (0, 1, 2, and 3 days) were subjected to single-cell RNA-seq at day 3 (FIG. 16A). They contained both mesoderm and endoderm populations. FIG. 16B depicts a feature plot of cell distributions under different BMP4 treatment durations. FIG. 16C depicts clustering of d3 meso/endoderm based on marker genes. FIG. 16D depicts a reference map comparison of d3 meso/endoderm to CS7 gastrulation human embryo AM: Advanced mesoderm; EM: Emergent mesoderm; PS: Primitive Streak. FIG. 16E depicts a cluster projection of day 3 meso/endoderm organoids to mouse early gastrulation embryos. i. UMAP of mouse E6.5˜E8.5 embryos. ii. Reverse projection of day 3 organoids to mouse embryos map. Accumulated stimulation by BMP4 augmented mesenchymal population, while reduced neural lineage (e.ii).

### Example 14: Characterization of Day 7 Vascularized Foregut and Mid/Hindgut Organoids by Single-Cell RNA-Seq

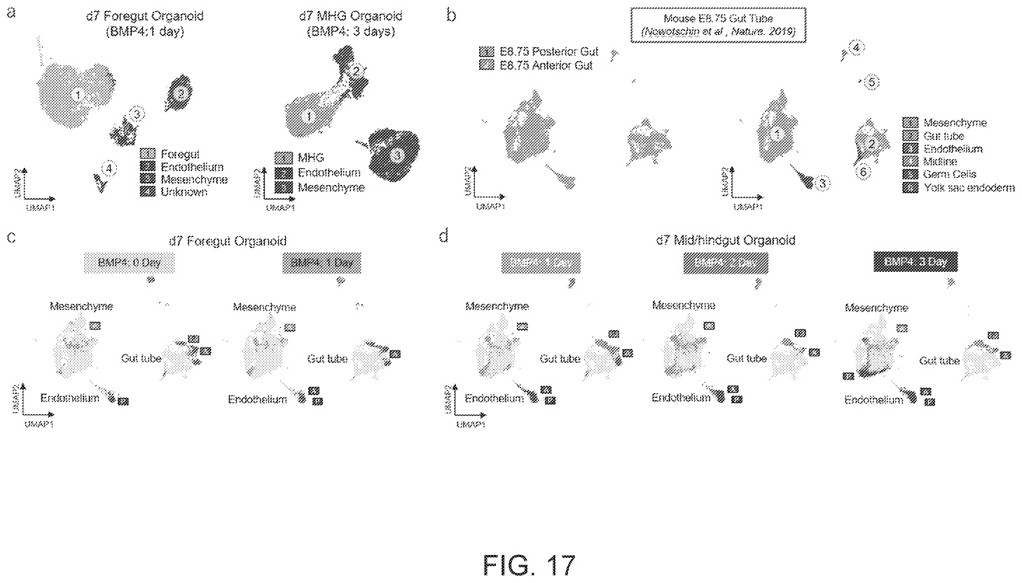

FIG. 17A depicts representative UMAP projection of foregut organoid (1-day treatment of BMP4 from day 1-3) and mid/hindgut organoid (3-days treatment of BMP4 from day 1-3). FIG. 17B-D depict reverse projection of human vascularized gut tube organoids to mouse E8.75 gut tube atlas. FIG. 17B depicts mouse E8.75 embryonic gut tube containing both anterior (A) and posterior (P) portion. Gut tube (endoderm), mesenchyme, and endothelium populations are segregated into A-P subclusters. FIG. 17C depicts Day 7 foregut organoid reverse projection to mouse E8.75 gut tube. Compared to no BMP4, treatment with one-day BMP4 generated purer anterior gut tube endoderm and more anterior endothelium. FIG. 17D depicts Day 7 mid/hindgut organoid reverse projection to mouse E8.75 tube. Longer treatment of BMP4 during the first three-day differentiation induced more posterior gut tube endoderm, mesenchyme, and endothelium.

### Example 15: Abnormal Phenotypes of vHLuO and vHIO from FOXF1-Mutant iPSC-Lines Accessed by Immunofluorescence Staining

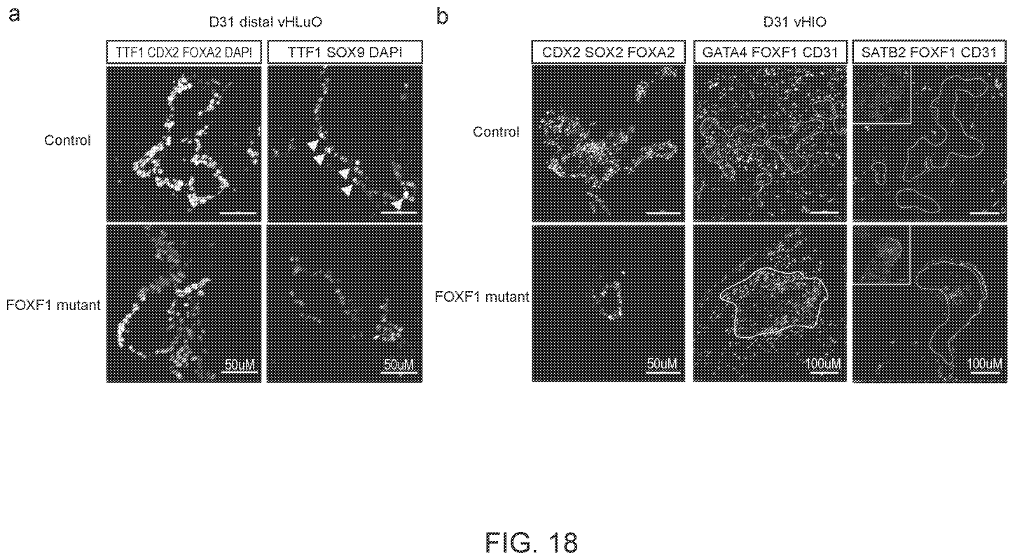

Compared to normal control (n=2), D31 distal vHLuO from FOXF1-mutant iPSC (n=2) showed mixed lung (TTF1+) and posterior gut tube (CDX2+) in epithelial structure (FOXA2+). Besides, FOXF1-distal vHLuO lost distal lung marker SOX9 expression. (FIG. 18A) Compared to normal control (n=2), D31 vHIO from FOXF1-mutant iPSCs (n=2) loss posterior gut tube marker (CDX2) with mixed anterior gut tube population (SOX2). Mesenchymal marker FOXF1 was also significantly suppressed. No clear expression difference of intestinal maker GATA4 or endothelium (CD31). Colonic marker SATB2 was slightly elevated in FOXF1-mutant vHIO. (FIG. 18B)

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed herein. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Barnes R M, et al. Analysis of the Hand1 cell lineage reveals novel contributions to cardiovascular, neural crest, extra-embryonic, and lateral mesoderm derivatives. Dev Dyn. 2010:239:3086-97.
Bolte C, et al. FOXF1 transcription factor promotes lung regeneration after partial pneumonectomy. Sci Rep. 2017; 7:10690.
Cakir B, et al. Engineering of human brain organoids with a functional vascular-like system. Nat Methods. 2019; 16:1169-1175.
Cao J, et al. A human cell atlas of fetal gene expression. Science. 2020:370.
Cardoso W V and Lu J. Regulation of early lung morphogenesis: questions, facts and controversies. Development. 2006; 133:1611-24.
D'Amour K A, et al. Efficient differentiation of human embryonic stem cells to definitive endoderm. Nat Biotechnol. 2005; 23:1534-41.
Davenport C, et al. Anterior-Posterior Patterning of Definitive Endoderm Generated from Human Embryonic Stem Cells Depends on the Differential Signaling of Retinoic Acid, Wnt-, and BMP-Signaling. Stem Cells. 2016; 34:2635-2647.
Gage B K, et al. Generation of Functional Liver Sinusoidal Endothelial Cells from Human Pluripotent Stem-Cell-Derived Venous Angioblasts. Cell Stem Cell. 2020; 27:254-269 e9.
Gonzales L W et al. Differentiation of human pulmonary type II cells in vitro by glucocorticoid plus cAMP. Am. J. Physiol. Lung Cell Mol. Physiol. 2002; 283:L940-L951.
Green M D, et al. Generation of anterior foregut endoderm from human embryonic and induced pluripotent stem cells. Nat Biotechnol. 2011; 29:267-72.
Gillich A, et al. Capillary cell-type specialization in the alveolus. Nature. 2020; 586:785-789.
Han L, et al. Single cell transcriptomics identifies a signaling network coordinating endoderm and mesoderm diversification during foregut organogenesis. Nat Commun. 2020:11:4158.
Hawkins F, et al. Prospective isolation of NKX2-1-expressing human lung progenitors derived from pluripotent stem cells. J. Clin. Invest. 2017; 127(6):2277-2294.
Holloway E M, et al. Differentiation of Human Intestinal Organoids with Endogenous Vascular Endothelial Cells. Dev Cell. 2020; 54:516-528 e7.
Homan K A, et al. Flow-enhanced vascularization and maturation of kidney organoids in vitro. Nat Methods. 2019; 16:255-262.
Jacob A, et al. Derivation of self-renewing lung alveolar epithelial type II cells from human pluripotent stem cells. Nature. 2019; 14:3303-3332.
Kalucka J, et al. Single-Cell Transcriptome Atlas of Murine Endothelial Cells. Cell. 2020:180:764-779 e20.
Lian X, et al. Robust cardiomyocyte differentiation from human pluripotent stem cells via temporal modulation of canonical Wnt signaling. Proc Natl Acad Sci USA. 2012; 109:E1848-57.
Little D R, et al. Differential chromatin binding of the lung lineage transcription factor NKX2-1 resolves opposing murine alveolar cell fates in vivo. Nat Commun. 2021; 12:2509.
Loh K M, et al. Efficient endoderm induction from human pluripotent stem cells by logically directing signals controlling lineage bifurcations. Cell Stem Cell. 2014; 14:237-52.
Loh K M, et al. Mapping the Pairwise Choices Leading from Pluripotency to Human Bone, Heart, and Other Mesoderm Cell Types. Cell. 2016; 166:451-467.
McCauley K B, et al. Efficient Derivation of Functional Human Airway Epithelium from Pluripotent Stem Cells via Temporal Regulation of Wnt Signaling. Cell Stem Cell. 2017; 20:844-857 e6.
McCauley K B, et al. Single-Cell Transcriptomic Profiling of Pluripotent Stem Cell-Derived SCGB3A2+ Airway Epithelium. Stem Cell Reports. 2018; 10:1579-1595.
McCracken K W, et al. Generating human intestinal tissue from pluripotent stem cells in vitro. Nat Protoc. 2011; 6:1920-8.
Miller A J, et al. Generation of lung organoids from human pluripotent stem cells in vitro. Nat Protoc. 2019; 14:518-540.
Miranda J, et al. A novel mutation in FOXF1 gene associated with alveolar capillary dysplasia with misalignment of pulmonary veins, intestinal malrotation and annular pancreas. Neonatology. 2013; 103:241-5.
Morrisey E E and Hogan B L. Preparing for the first breath: genetic and cellular mechanisms in lung development. Dev Cell. 2010; 18:8-23.
Mou H, et al. Generation of multipotent lung and airway progenitors from mouse ESCs and patient-specific cystic fibrosis iPSCs. Cell Stem Cell. 2012; 10:385-97.
Munera J O, et al. Differentiation of Human Pluripotent Stem Cells into Colonic Organoids via Transient Activation of BMP Signaling. Cell Stem Cell. 2017; 21:51-64 e6.
Nguyen J, et al. The next generation of endothelial differentiation: Tissue-specific ECs. Cell Stem Cell. 2021; 28:1188-1204.
Paik D T, et al. Single-Cell RNA Sequencing Unveils Unique Transcriptomic Signatures of Organ-Specific Endothelial Cells. Circulation. 2020:142:1848-1862.
Palikuqi B, et al. Adaptable haemodynamic endothelial cells for organogenesis and tumorigenesis. Nature. 2020; 585:426-432.
Peng T, et al. Coordination of heart and lung co-development by a multipotent cardiopulmonary progenitor. Nature. 2013; 500:589-92.
Penkala I J, et al. Age-dependent alveolar epithelial plasticity orchestrates lung homeostasis and regeneration. Cell Stem Cell. 2021.
Pradhan A, et al. The S52F FOXF1 Mutation Inhibits STAT3 Signaling and Causes Alveolar Capillary Dysplasia. Am J Respir Crit Care Med. 2019; 200:1045-1056.
Rodrigues Toste de Carvalho A L, et al. The in vitro multilineage differentiation and maturation of lung and airway cells from human pluripotent stem cell-derived lung progenitors in 3D. Nat Protoc. 2021; 16:1802-1829.
Takebe T, et al. Vascularized and functional human liver from an iPSC-derived organ bud transplant. Nature. 2013; 499:481-4.
Ustiyan V, et al. FOXF1 transcription factor promotes lung morphogenesis by inducing cellular proliferation in fetal lung mesenchyme. Dev Biol. 2018; 443:50-63.
Vila Ellis L, et al. Epithelial Vegfa Specifies a Distinct Endothelial Population in the Mouse Lung. Dev Cell. 2020; 52:617-630 e6.
Wimmer R A, et al. Generation of blood vessel organoids from human pluripotent stem cells. Nat Protoc. 2019; 14:3082-3100.
Yu Q, et al. Charting human development using a multi-endodermal organ atlas and organoid models. Cell. 2021; 184:3281-3298 e22.
Zhang S, et al. Vascularized organoids on a chip: strategies for engineering organoids with functional vasculature. Lab Chip. 2021; 21:473-488.
Zorn A M and Wells J M. Vertebrate endoderm development and organ formation. Annu Rev Cell Dev Biol. 2009; 25:221-51.

1. A method of producing mesendoderm spheroids comprising both definitive endoderm and lateral plate mesoderm, comprising:
a) activating a TGF-b pathway, a BMP pathway, and a Wnt pathway in pluripotent stem cells for about 24 hours to about 48 hours; and
activating a TGF-b pathway and a BMP pathway, without activating a Wnt pathway, in the cells of step a), for about 24 hours to about 72 hours;
thereby differentiating the pluripotent stem cells to mesendoderm spheroids comprising both FOXA2+ definitive endoderm and HAND1+ lateral plate mesoderm;
wherein the cells of step a) and/or b) are contacted with a serum supplement, optionally fetal bovine serum.

2. The method of claim 1, wherein the lateral plate mesoderm surrounds the definitive endoderm in the mesendoderm spheroids.

3. The method of claim 1, wherein step a) is performed for about 26, 27, 28, 29, or 30 hours, optionally about 28 hours.

4. The method of claim 1, wherein step b) is performed for about 42, 43, 44, 45, 46, 47, 48, 49, 50, 51, 52, 53, 54, 55, 56, 57, 58, 59, or 60 hours, optionally about 44 hours.

5. The method of claim 1, wherein activating the TGF-b pathway in step a) and/or b) comprises providing a TGF-b activator; optionally wherein the TGF-b activator is TGF-beta 1, TGF-beta 2, TGF-beta 3, Activin A, Activin B, Nodal, a BMP, IDE1, IDE2, or any combination thereof, optionally Activin A.

6. The method of claim 5, wherein the TGF-b pathway activator of step a) and/or b) is provided at a concentration of about 50, 60, 70, 80, 90, 100, 110, 120, 130, 140, or 150 ng/mL, or any concentration within a range defined by any two of the aforementioned concentrations.

7. The method of claim 1, wherein activating the BMP pathway in step a) and/or b) comprises providing a BMP activator; optionally wherein the BMP activator is BMP1, BMP2, BMP3, BMP4, BMP5, BMP6, BMP7, BMP8a, BMP8b, BMP10, BMP11, BMP15, IDE1, IDE2, or any combination thereof, optionally BMP4.

8. The method of a claim 7, wherein the BMP pathway activator of step a) and/or b) is provided at a concentration of about 10, 20, 30, 40, 50, 60, 70, 80, 90, or 100 ng/mL, or any concentration within a range defined by any two of the aforementioned concentrations.

9. The method of claim 1, wherein activating the Wnt pathway comprises providing a Wnt pathway activator; optionally wherein the Wnt pathway activator is Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, Wnt16, BML 284, IQ-1, WAY 262611, CHIR99021, CHIR 98014, AZD2858, BIO, AR-A014418, SB 216763, SB 415286, aloisine, indirubin, alsterpaullone, kenpaullone, lithium chloride, TDZD 8, TWS119, or any combination thereof, optionally CHIR99021.

10. The method of claim 9, wherein the Wnt pathway activator is provided at a concentration of about 1, 2, 3, 4, 5, 6, 7, 8, 9, 10, 11, 12, 13, 14, 15, 16, 17, 18, 19, 20, 21, 22, 23, or 24 μM, or any concentration within a range defined by any two of the aforementioned concentrations, optionally 9, 10, 11, or 12 μM.

11. The method of claim 1, wherein prior to the contacting of step a), the pluripotent stem cells are in the form of spheroids.

12. The method of claim 11, wherein the pluripotent stem cells are formed into spheroids by aggregation, optionally in an aggregation well, optionally wherein the number of cells aggregated is, or is about 200-4000, 300-3000, 500-2000, 600-1500, or 750-1250 cells.

13. The method of claim 1, wherein for the duration of steps a) and b), the pluripotent stem cells are kept in suspension, optionally with shaking, and/or not as a monolayer.

14. The method of claim 1, wherein the FOXA2+ definitive endoderm and the HAND1+ lateral plate mesoderm are approximately at a 1:1 ratio.

15. Mesendoderm spheroids prepared according to the method of claim 1.

16.-85. (canceled)

86. A mesendoderm spheroid, comprising both definitive endoderm and lateral plate mesoderm.

87. The mesendoderm spheroid of claim 86, comprising both FOXA2+ definitive endoderm and HAND1+ lateral plate mesoderm.

88. The mesendoderm spheroid of claim 87, wherein the FOXA2+ definitive endoderm and HAND1+ lateral plate mesoderm are approximately at a 1:2, 1:1.5, 1:1, 1.5:1, or 2:1 ratio.

89. The mesoderm spheroid of claim 86, wherein the lateral plate mesoderm surrounds the definitive endoderm in the mesoderm spheroid.

90. The mesendoderm spheroid of claim 86, wherein the mesendoderm spheroid comprises a FOXF1 mutation.
